(19) United States
(12) Patent Application Publication
(10) Pub. No.: US 2026/0290610 A1
(43) Pub. Date: Sep. 24, 2026

(54) SYSTEM

(71) Applicant: SoftBank Group Corp., Tokyo (JP)

(72) Inventor: Hiroma SAGO, Tokyo (JP)

(73) Assignee: SoftBank Group Corp., Tokyo (JP)

(21) Appl. No.: 19/567,272

(22) Filed: Mar. 16, 2026

(30) Foreign Application Priority Data
Mar. 19, 2025 (JP) 2025-045243

(51) Int. Cl. G16H 50/30; G06N 3/0475; G16H 20/70

(52) U.S. Cl. CPC G16H 50/30; G06N 3/0475; G16H 20/70

(57) ABSTRACT

A system comprising a processor, wherein the processor is configured to: input personal health information and an emotional state of an individual, analyze the personal health information and the emotional state of the individual, generate prompts to instruct generation of proposals of diet and medication based on preventive medicine, provide the generated proposals to the individual, enable ordering by cooperating with an external food delivery service, generate prompts to instruct adjustment of alcohol intake restrictions based on the emotional state of the individual, and adjust the alcohol intake restrictions using the prompts.

### CROSS-REFERENCE TO RELATED APPLICATION

This application is based on and claims priority under 35 USC 119 from Japanese Patent Application No. 2025-045243 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional health management systems typically provide static recommendations based solely on personal health information such as age, weight, medical history, and lifestyle. These systems generally do not consider an individual's emotional state in real time, and therefore fail to adapt dietary and medication-related proposals to changes in mood or stress level. As a result, users may receive recommendations that are medically reasonable but poorly matched to their psychological condition, which can reduce adherence to preventive medicine guidance. Furthermore, conventional systems that suggest foods to be eaten usually stop at providing textual advice and do not integrate with external food delivery services to enable immediate ordering of recommended foods. In addition, such systems do not dynamically adjust alcohol intake restrictions based on the individual's emotional state, which can lead to insufficient control of risky drinking behavior under stress or negative emotions. Accordingly, there is a need for a system that integrates personal health information and emotional state, generates preventive-medicine-based proposals including diet, medication, and stress management, cooperates with external food delivery services to enable ordering of recommended foods, and dynamically adjusts alcohol intake restrictions in accordance with the individual's emotional state.

### SUMMARY

In order to solve the above-described problems, a system is provided comprising a processor, wherein the processor is configured to input personal health information and an emotional state of an individual, analyze the personal health information and the emotional state of the individual, and generate prompts to instruct generation of proposals of diet and medication based on preventive medicine. The processor is further configured to provide the generated proposals to the individual so that the individual can receive personalized guidance reflecting both objective health conditions and subjective emotional status. In addition, the processor is configured to enable ordering by cooperating with an external food delivery service, thereby allowing the individual to directly order foods corresponding to the generated proposals through integration with an external ordering platform. Moreover, the processor is configured to generate prompts to instruct adjustment of alcohol intake restrictions based on the emotional state of the individual, and to adjust the alcohol intake restrictions using the prompts so that alcohol consumption limits can be dynamically tightened or relaxed in response to changes in emotional state. The processor may also be configured to generate stress management proposals and generate foods that are recommended to be eaten based on the personal health information and the emotional state of the individual, and to enable ordering of the generated foods by cooperating with the external food delivery service.

The term “system” refers to an apparatus including at least one processor and, optionally, one or more memories, communication interfaces, input devices, and output devices, which cooperatively perform the functions described in the present specification and claims.

The term “processor” refers to any hardware component or set of components capable of executing instructions, including but not limited to a central processing unit (CPU), a microprocessor, a microcontroller, a graphics processing unit (GPU), a digital signal processor (DSP), or a combination thereof, whether implemented as a single chip or as multiple distributed processing units.

The term “individual” refers to a human user whose personal health information and emotional state are acquired, analyzed, and used as a basis for generating proposals, adjustments, and other outputs in the system.

The term “personal health information” refers to information relating to the physical or medical state of the individual, including, for example, age, sex, weight, height, medical history, current diseases, medications, allergies, genetic information, lifestyle, and other data relevant to preventive medicine or health management.

The term “emotional state” refers to information representing the psychological or affective condition of the individual at a given time, including, for example, feelings such as stress, anxiety, happiness, sadness, anger, or calmness, which may be obtained through user input, sensor data, behavioral analysis, or other measurement methods.

The term “analyze” refers to a process in which the processor processes and evaluates the personal health information and the emotional state, using algorithms, rules, models, or statistical methods, in order to derive relationships, risk assessments, or conditions that are used to generate proposals, prompts, and adjustments.

The term “preventive medicine” refers to a field of medicine focused on preventing disease, maintaining health, and reducing risk factors, and includes dietary guidance, medication management, lifestyle modification, and stress management intended to prevent the onset or progression of diseases.

The term “proposal” refers to information generated by the processor as a recommendation or guidance for the individual, including, for example, suggestions related to diet, medication, stress management, or other health-related behaviors.

The term “diet proposal” refers to a proposal relating to foods, meals, or nutritional patterns that are recommended for the individual based on preventive medicine, the personal health information, and the emotional state.

The term “medication proposal” refers to a proposal relating to the use, adjustment, or management of medications in view of preventive medicine, including, for example, recommendations to consult a physician, suggestions regarding adherence to prescribed medications, or general guidance concerning medication categories, while not necessarily specifying particular drug products.

The term “stress management proposal” refers to a proposal that suggests methods or behaviors for reducing, coping with, or preventing stress, including, for example, relaxation techniques, exercise, sleep improvement, behavioral therapy guidance, or recommendations to seek professional support.

The term “prompt” refers to a control signal, instruction, message, or data structure generated by the processor to trigger, control, or guide a subsequent process, such as generation of proposals or adjustment of alcohol intake restrictions, either within the processor or in an external system.

The term “generate prompts” refers to an operation in which the processor creates prompts as defined above, based on the analyzed personal health information and emotional state, for use in instructing the generation of proposals or the adjustment of restrictions.

The term “provide” refers to an operation in which the processor outputs or causes to be output information, such as proposals or prompts, to the individual through a user interface, display device, communication device, or any other output mechanism.

The term “external food delivery service” refers to a service or system that is separate from the claimed system and that receives food orders, processes such orders, and facilitates preparation and delivery of foods to the individual, typically via a network-based ordering platform.

The term “cooperating with an external food delivery service” refers to an operation in which the processor communicates or exchanges data with the external food delivery service, for example by sending order information, menu selections, or identifiers of recommended foods, so that foods corresponding to proposals generated by the system can be ordered.

The term “enable ordering” refers to a function in which the processor allows the individual to place an order for foods corresponding to proposals generated by the system, by using communication with the external food delivery service, and may include generating order data, transmitting order data, and handling confirmations.

The term “alcohol intake restriction” refers to a limit or guideline concerning the amount, frequency, or conditions of alcohol consumption by the individual, which may be represented as numerical thresholds, time-based conditions, or policy rules.

The term “adjustment of alcohol intake restrictions” refers to a modification of the alcohol intake restriction, including tightening or relaxing limits, changing allowable times or quantities, or imposing temporary bans, in response to changes in the emotional state or other conditions.

The term “foods that are recommended to be eaten” refers to specific food items, meal compositions, or menus that the processor identifies as appropriate for consumption by the individual based on the personal health information, the emotional state, and preventive medicine principles.

The term “using the prompts” refers to an operation in which the processor applies or executes the instructions or control information included in the prompts, for example to control internal processing for generating proposals, or to modify parameters defining alcohol intake restrictions.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### FIRST EXEMPLARY EMBODIMENT

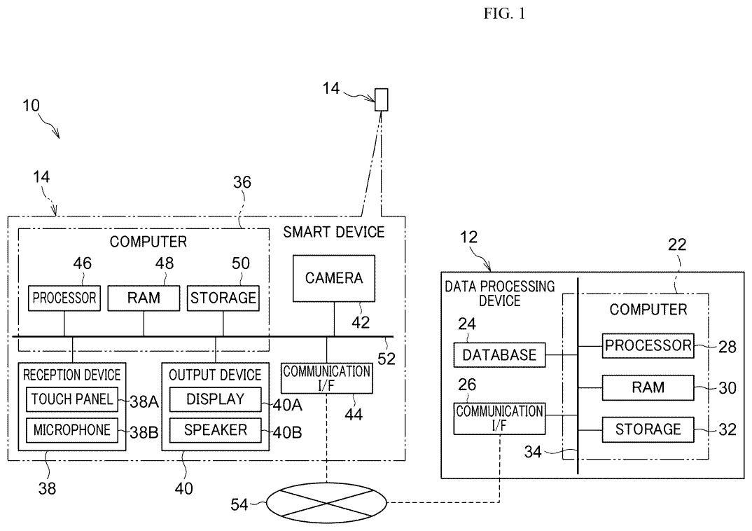

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

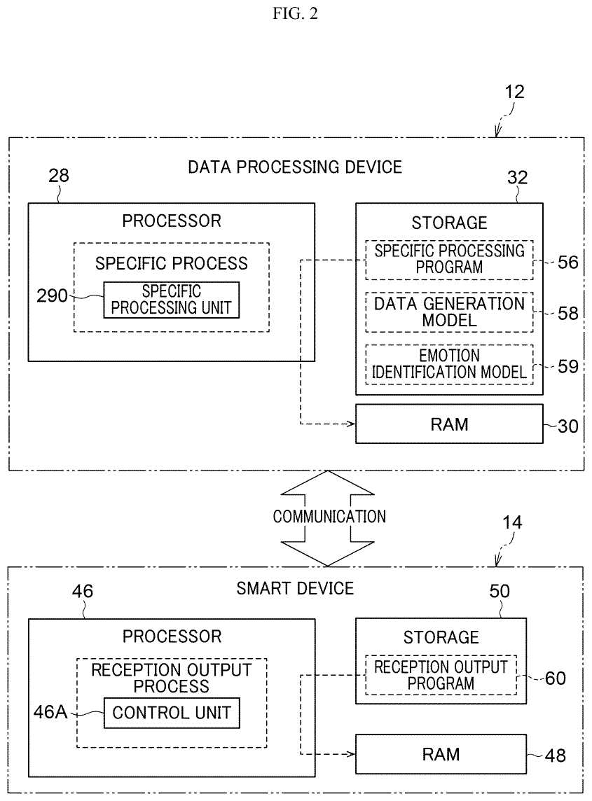

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional health advisory systems typically implement fixed rule sets or static recommendation engines on general-purpose computer hardware. Such systems often perform only simple threshold checks on biometric values and emotional status indicators and then output generic messages. As a result, the underlying computer resources, including processors, storage, and communication interfaces, are not utilized to generate context-rich machine interpretable prompts or to orchestrate dynamic interactions with external machine learning services. This leads to several technical problems in the field of computer technology.

First, conventional systems do not efficiently transform heterogeneous, user-specific health-related and emotional-related information into structured representations that are suitable for high-value downstream processing. Health data, lifestyle attributes, and emotional indicators are often stored as loosely structured records without systematic statistical computation or normalization. Consequently, subsequent processing modules are forced either to re-parse unstructured data or to ignore contextual relationships, causing redundant computations, increased latency, and inefficient memory usage in the processing pipeline.

Second, known systems do not exploit generative artificial intelligence models in a manner that is tightly integrated into the system architecture. When generative models are used, they are typically invoked with manually crafted, ad hoc input sentences, which are not derived from machine-executed statistical computations. This ad hoc approach results in inconsistent prompt generation, redundant network calls, and underutilization of the processing capabilities of both the local server and the external generative AI infrastructure. The lack of an automated prompt generation mechanism, grounded in computed health indices and risk indices, prevents the system from optimizing the number and content of external inference requests, thereby increasing network traffic and processing time.

Third, conventional systems generally separate user-facing advice generation from external service orchestration, such as cooperation with goods delivery services. The computer system often treats recommendation generation and ordering control as independent applications, which introduces duplicated data transformations, repeated parsing of natural language outputs, and fragmented state management between modules. This fragmented architecture causes unnecessary processor cycles and memory allocation for converting between human-readable text and machine-actionable order information. It also complicates error handling and leads to inefficiencies in managing the life cycle of advisory and ordering transactions.

Fourth, known systems do not implement a feedback-driven, dynamic control mechanism for constraints such as alcohol intake limitations based on evolving emotional-related information and up-to-date statistical health analysis. Typical implementations apply static limits or rely on user self-reporting without re-computing constraints using updated emotional states and computed indices. This absence of a closed-loop, machine-controlled adjustment mechanism results in inflexible system behavior and fails to leverage the processor's ability to repeatedly generate, refine, and execute prompt sentences that guide a generative AI model to compute updated control parameters.

Accordingly, there is a need for a computer-implemented system that (i) acquires and statistically processes health-related and emotional-related information into structured indices on a server, (ii) automatically generates structured prompt sentences for a generative AI model based on those indices, (iii) programmatically extracts machine-actionable proposals from the AI-generated natural language responses, (iv) integrates the proposals with external transaction services such as goods delivery systems, and (v) dynamically updates consumption constraints, such as alcohol intake restrictions, based on continuous interaction between the server, the user terminal, and the generative AI model. By addressing these technical problems, the invention aims to improve the efficiency, scalability, and functional capabilities of computer systems that manage personalized health and lifestyle control workflows.

The specific processing by the specific processing unit 290 of the data processing device 12 in Example 1 is realized by the following means.

The present invention provides a server comprising one or more processors configured to acquire individual health-related information and emotional-related information via a user interface of an information processing terminal and to transmit the individual health-related information and the emotional-related information through a communication network to the server; to store the transmitted health-related information and emotional-related information as structured information in a storage device; to perform statistical computation processing on at least the stored health-related information to generate an analysis result including at least a body index, a risk index, and a lifestyle index; to automatically generate, based on the analysis result, a machine-readable prompt sentence for a generative AI model, the prompt sentence describing in natural language instructions related to at least nutrient intake, ingestion of substances, execution of physical activity, improvement of lifestyle habits, and adjustment of emotional states; to transmit the prompt sentence as a response generation request to an external generative AI model and to receive a natural language response from the external generative AI model via the communication network; to parse the natural language response and extract therefrom at least a meal proposal, an ingestion substance proposal, a physical activity proposal, a stress management proposal, and an alcohol intake restriction proposal conditioned on the emotional-related information; to convert the extracted proposals into display information for presentation on the information processing terminal; to generate cooperation information, based on at least the meal proposal included in the display information, for enabling selection and ordering of proposed food items by performing communication with an external goods delivery service; and to additionally generate, based on at least the emotional-related information and the analysis result, a further prompt sentence for a generative AI model regarding relaxation or strengthening of alcohol intake restrictions and to dynamically update, using a response from the generative AI model, control data that defines the alcohol intake restrictions applied to the individual. This enables a computer system to more efficiently transform heterogeneous health-related and emotional-related inputs into structured indices, to automatically generate and manage context-rich prompt sentences for interaction with a generative AI model, to reduce redundant parsing and data conversion between advisory output and ordering control, and to implement a closed-loop, dynamically adjustable constraint control mechanism that leverages both local statistical computation and remote generative inference, thereby improving overall processing efficiency, scalability, and functional integration in the health advisory and lifestyle management workflow.

The term “individual health-related information” refers to data associated with a particular person's physical condition, medical history, biometric measurements, lifestyle habits, and other attributes relevant to health assessment and management, including but not limited to age, sex, body size, past illnesses, allergies, and exercise patterns.

The term “emotional-related information” refers to data indicating a person's emotional or psychological state, including but not limited to reported mood, stress level, anxiety level, affective tendencies, and other indicators of mental or emotional status.

The term “information processing terminal” refers to an electronic apparatus operated by a user, such as a portable communication device, a personal computing device, or a stationary display device, that is capable of presenting a user interface and exchanging data with a server over a communication network.

The term “user interface” refers to a hardware and software configuration, including graphical, textual, or auditory components, that enables a user to input information, receive information, and interact with an application running on an information processing terminal or server.

The term “communication network” refers to a wired or wireless data transmission infrastructure, including local networks and wide area networks, that enables exchange of digital information between an information processing terminal, a server, and external services.

The term “information processing apparatus” refers to a computing device or a group of computing devices, including at least one processor and at least one storage device, configured to execute programs, process data, and control communication with other devices.

The term “storage device” refers to a physical or virtual memory component, such as a semiconductor memory, a magnetic or optical storage medium, or a cloud-based storage resource, that is configured to store data and program instructions in a non-transitory manner or a transient manner.

The term “structured information” refers to data that is stored or represented in a predetermined, machine-readable format, such as tables, records, key-value sets, or other organized data structures, suitable for systematic retrieval and processing.

The term “statistical computation processing” refers to a series of programmed operations that apply mathematical, statistical, or analytical techniques to numerical or categorical data to generate derived values, indices, classifications, or other quantitative or qualitative metrics.

The term “analysis result” refers to information generated by statistical computation processing or other analytical operations that summarizes or evaluates input data, and that includes at least one computed index, risk level, or category used by subsequent processing steps.

The term “body index” refers to a numerical or categorical indicator derived from physical measurements of a person, such as body mass index, weight-to-height ratio, or other indices that characterize body composition or size.

The term “risk index” refers to a numerical or categorical indicator that represents a likelihood or severity of a health-related event or condition, calculated on the basis of health-related information, lifestyle factors, and analysis rules.

The term “lifestyle index” refers to a numerical or categorical indicator that reflects aspects of a person's daily habits, such as exercise frequency, dietary pattern, sleep pattern, and substance use, and that is derived from lifestyle-related data.

The term “prompt sentence” refers to one or more machine-generated natural language expressions that are structured to serve as input for a generative AI model, and that provide contextual information and instructions to guide generation of a response.

The term “generative AI model” refers to a machine learning model, such as a neural network-based language model, configured to generate text or other output data in response to an input prompt, based on parameters learned from training data.

The term “response generation request” refers to a message or data structure transmitted from a server to a generative AI model that contains at least a prompt sentence and optional parameters, and that instructs the generative AI model to generate a corresponding response.

The term “natural language response” refers to one or more text segments expressed in a human language and generated by a generative AI model in response to a prompt sentence.

The term “meal proposal” refers to information included in a natural language response that suggests food items, menus, or dietary plans appropriate for a person's health-related condition and lifestyle.

The term “ingestion substance proposal” refers to information included in a natural language response that suggests ingestible substances, such as nutrients, supplements, or other consumable materials, which may be appropriate for a person's health-related condition.

The term “physical activity proposal” refers to information included in a natural language response that suggests activities involving bodily movement, such as exercise routines, daily movement goals, or training plans.

The term “stress management proposal” refers to information included in a natural language response that suggests actions, behaviors, or techniques intended to reduce or manage psychological stress.

The term “alcohol intake restriction proposal” refers to information included in a natural language response that specifies limits, conditions, or guidelines regarding consumption of alcoholic beverages by a person.

The term “display information” refers to data that has been formatted for presentation on a user interface of an information processing terminal, including textual, graphical, or other visual elements.

The term “cooperation information” refers to data generated by a server to enable or control interaction between the server and an external service, including identifiers, item selections, order parameters, and other data necessary for coordinated processing.

The term “external goods delivery service” refers to an information processing system operated by a third party that receives order-related data via a communication network and arranges procurement, preparation, and delivery of goods, including food items.

The term “food items” refers to edible products intended for human consumption, including ingredients, prepared dishes, beverages, and packaged foods.

The term “control data” refers to machine-readable information used by a server or terminal to govern or constrain behavior, limits, or parameters of a function, such as numerical thresholds or categorical rules for alcohol intake.

The term “alcohol intake restrictions” refers to computationally maintained limits or conditions regarding the amount, frequency, or type of alcoholic beverage that a person is permitted or recommended to consume, as determined or updated by a system.

The term “dynamic update” refers to a process in which stored data, parameters, or control values are modified over time in response to new input information, computation results, or external responses, without requiring system restart or manual reconfiguration.

The term “machine-readable” refers to a form of data or text that is structured so that it can be programmatically generated, stored, transmitted, and interpreted by a computing device without requiring human intervention for basic understanding of its structure or meaning.

In one embodiment, a server cooperates with a terminal operated by a user to implement a health advisory and lifestyle management system that utilizes a generative AI model. The server comprises one or more processors, a main memory, a non-transitory storage device, and a communication interface connected to a communication network. The terminal comprises a processor, a memory, a display, input devices such as a touch panel or keyboard, and a communication module.

The terminal executes a web browser or a dedicated application running on an operating system such as a mobile operating system or a desktop operating system. The terminal presents a user interface that includes input fields for individual health-related information and emotional-related information. The user operates the terminal to input age, sex, height, weight, past illnesses, allergies, lifestyle patterns, and subjective or measured emotional states such as stress level or mood. The terminal converts the input into a structured internal representation and transmits the representation to the server via the communication network using a secure communication protocol.

The server receives the transmitted data and stores the data in the storage device, which can be implemented by a relational database management system operating on magnetic storage or solid-state storage. The server normalizes the health-related information into a structured format that includes, for example, a height value in meters, a weight value in kilograms, a code list for past illnesses, a code list for allergies, and categorical values for lifestyle and emotional states. The server stores the structured information as records in database tables with defined schemas. This structured storage enables index-based retrieval and reduces the need for repeated parsing, thereby improving data management efficiency.

The server executes an analysis program implemented, for example, in a high-level programming language running on a runtime environment such as a Python interpreter. The server loads the structured records into an internal data structure that is equivalent to a table with named fields. The server performs statistical computation processing on the records to calculate a body index, such as a body mass index, and additional indices such as a cardiovascular risk index and a lifestyle index. The server calculates the indices by applying numerical formulas and rule-based evaluations to the values stored in the structured records. The server stores the computed indices in memory as part of an analysis result object associated with the user.

The server generates a prompt sentence for a generative AI model based on the analysis result. The server assembles the prompt sentence by inserting computed indices and categorized risk levels into predefined sentence templates. The server uses the structured indices rather than raw user input, which reduces variability and improves the specificity of the generated prompt sentence. By generating the prompt sentence from machine-computed indices, the server ensures that similar health states result in similar prompt structures, which stabilizes the behavior of the generative AI model and reduces unnecessary variance in responses.

In one example, the server generates the following prompt sentence:
“This user is a 45-year-old male with a height of 172 cm and a weight of 68 kg, which corresponds to a BMI of 22.9 in the normal range. The user has a past medical history of hypertension and a family history of diabetes. The user's lifestyle includes a high-salt diet, low exercise frequency, and alcohol consumption three times per week. Based on these factors, the server's statistical analysis estimates the user's cardiovascular risk level as moderate. Using this information, generate clear, personalized health advice in English.

Focus on specific and practical recommendations for diet (especially reducing salt and overall cardiovascular risk), exercise (including weekly duration and intensity), and general lifestyle habits. Explain why each recommendation is beneficial, in terms that a non-medical professional can easily understand.”

In another example, the server generates a prompt sentence focused on obesity and diabetes risk:
“This user is a 35-year-old female with a height of 160 cm and a weight of 80 kg, resulting in a BMI of 31.3 in the obese range. She has a past medical history of gestational diabetes and currently exercises about once per week. She drinks sweet beverages every day and works at a desk job. She has a drug allergy that must be taken into account in any recommendations.

Assess this user's risks for type 2 diabetes and cardiovascular disease in qualitative terms, and provide specific, practical advice on diet, exercise, and lifestyle changes that are safe given the reported allergy. Include both short-term steps within 1-3 months and longer-term goals within 6-12 months.”

The server transmits the generated prompt sentence to a generative AI model provided by an external system. The server communicates with the external system via the communication interface using an application programming interface. The generative AI model is implemented as a neural network of the transformer type, comprising multiple encoder-decoder or decoder-only layers, self-attention mechanisms, and feedforward sublayers. The model stores learned parameters representing weights for attention, intermediate projections, and output projections. The model has been trained in advance on large-scale text datasets using a supervised or self-supervised learning method, in which the model minimizes an error function such as a cross-entropy loss over predicted tokens relative to target tokens. During training, an optimization algorithm such as a variant of stochastic gradient descent updates the weights by backpropagating gradients of the error function. The training process may include data augmentation techniques such as token masking, dropout within layers, or input perturbation to improve generalization.

The server does not retrain the generative AI model in this embodiment but uses the model in an inference mode. The server transmits the prompt sentence and control parameters such as maximum output length and randomness parameters to the external system. The external system receives the prompt sentence, encodes the sentence into token embeddings, and propagates the embeddings through the transformer layers to generate a sequence of output tokens that form a natural language response. The external system returns the response to the server.

The server receives the natural language response and parses the response. The server executes a response-processing module that identifies sections corresponding to meal proposals, ingestion substance proposals, physical activity proposals, stress management proposals, and alcohol intake restriction proposals. The server performs this identification by applying pattern-based or classification-based processing to the response text. For example, the server detects section headers or keywords and associates sentences with corresponding proposal categories. The server converts the identified proposals into an internal structured representation. This structured representation allows the server to use the proposals as control data for external services and for internal constraint management.

The server uses the meal proposals to generate cooperation information for an external goods delivery service. The server maps food names described in the natural language response to product identifiers stored in a product database or obtained from the external service. The server constructs an order candidate list including product identifiers, quantities, and delivery options, and transmits the order candidate list to the external goods delivery service. The external goods delivery service responds with availability information, prices, and delivery time options. The server stores this information and converts it into display information that the terminal can present to the user as selectable order options. By automatically transforming natural language meal proposals into machine-actionable order candidates, the server reduces repeated parsing at the terminal and minimizes redundant network transactions between multiple separate applications.

The terminal receives the display information and presents the advisory content and order options to the user. The terminal displays the analysis results, such as body index and risk index, alongside the natural language recommendations and selectable meal options derived from the external goods delivery service. The user may select certain meal options or follow the exercise and stress management advice presented on the display.

The server additionally uses emotional-related information and updated analysis results to control alcohol intake restrictions. The server maintains control data representing current recommended limits for alcohol consumption, such as maximum number of servings per day or per week. The server periodically or upon receipt of new emotional-related information recomputes relevant indices and generates a further prompt sentence describing the user's health state, emotional state, and current alcohol intake restriction. The server includes in the prompt sentence a request for adjustment recommendations, which may propose relaxation or strengthening of the restrictions.

In one example, the server generates the following further prompt sentence:
“This user currently has a recommended limit of two alcoholic drinks per day and ten per week, based on a moderate cardiovascular risk and a history of hypertension. Over the past week, the user has reported increased stress and poor sleep, but no acute medical symptoms.

The most recent analysis shows no significant change in body index but a slight increase in reported anxiety. Based on this health status and emotional state, evaluate whether the current alcohol intake restrictions should be relaxed, maintained, or strengthened. Provide updated recommended limits in terms of maximum drinks per day and per week, and explain the rationale.”

The generative AI model returns an updated recommendation. The server extracts the updated numerical limits and writes them into the control data in the storage device. The server then uses this updated control data to govern subsequent proposals related to alcohol intake and to control which recommendations are shown to the user. The dynamic update mechanism allows the server to close a feedback loop between new health-related and emotional-related inputs and the constraints enforced by the system.

The server achieves technical improvements in multiple respects. The server reduces communication load by generating prompt sentences that embed multiple structured indices in a single, context-rich request, thereby reducing the number of round-trip interactions with the external generative AI system. The server improves processing speed by storing normalized, structured information and computed indices in a database and in memory, which eliminates redundant computations and parsing for each interaction. The server improves accuracy of advisory content by conditioning prompt sentences on consistently computed indices rather than on raw, unnormalized input, which stabilizes the behavior of the generative AI model. The server reduces memory usage and error rates in downstream processing by converting natural language responses into structured proposals that are directly usable by external goods delivery services without manual re-interpretation.

The server implements non-conventional processing sequences and data structures that go beyond simple automation of human judgment. The server defines internal data objects for health indices, risk indices, lifestyle indices, and constraint parameters and uses these objects to generate machine-readable prompt sentences and to update control data. The server does not simply mirror a human expert's narrative process but executes rule-based and numerical computations that consistently transform heterogeneous inputs into stable features for the generative AI model. By designing the prompt generation module to operate directly on computed indices, the server improves the computational efficiency and reduces variability in AI outputs, which is a computer-centric improvement rather than a mere business process optimization.

In one alternative embodiment, the server executes the analysis program and the prompt generation program within a containerized environment managed by a container orchestration platform. The server distributes computation across multiple processor cores to parallelize the statistical computation processing for batches of users. The server aggregates multiple users' data into mini-batches and computes indices in vectorized form, thereby improving throughput and reducing latency.

In another embodiment, the generative AI model is deployed within a local inference server under control of the operator of the system. The server executes the generative AI model on dedicated acceleration hardware such as a graphics processing unit. The server streams token sequences to the local inference server and receives partial outputs incrementally. The server analyzes partial outputs to pre-fetch mapping data from the external goods delivery service or to pre-allocate display regions at the terminal, further reducing total response time.

In another variation, the server uses a hybrid approach combining rule-based selection and generative AI outputs. The server first applies deterministic rules to the analysis result to identify certain safety constraints, such as absolute contraindications for specific food items or alcohol levels based on medical conditions. The server then incorporates these constraints into the prompt sentence and instructs the generative AI model to generate recommendations that comply with the constraints. The server verifies the response against the constraints and discards or modifies recommendations that violate the constraints before presenting them to the user. This combination of rule-based and generative processing provides improved safety and reliability while still benefiting from the generative model's ability to produce detailed, personalized advice.

In all these embodiments, the server structures data flow among distinct modules, including acquisition, normalization, statistical computation, prompt generation, external model interaction, response parsing, constraint updating, and external service cooperation. The specific internal data structures and processing order are selected to improve the functioning of the computer system itself, including improved memory access patterns, reduced redundant computations, and optimized communication with external systems. The terminal and the server cooperate to present the generated advice and order options to the user in real time or near real time, enabling the user to apply the recommendations in everyday life, while the system maintains efficient and technically improved processing within the computing environment.

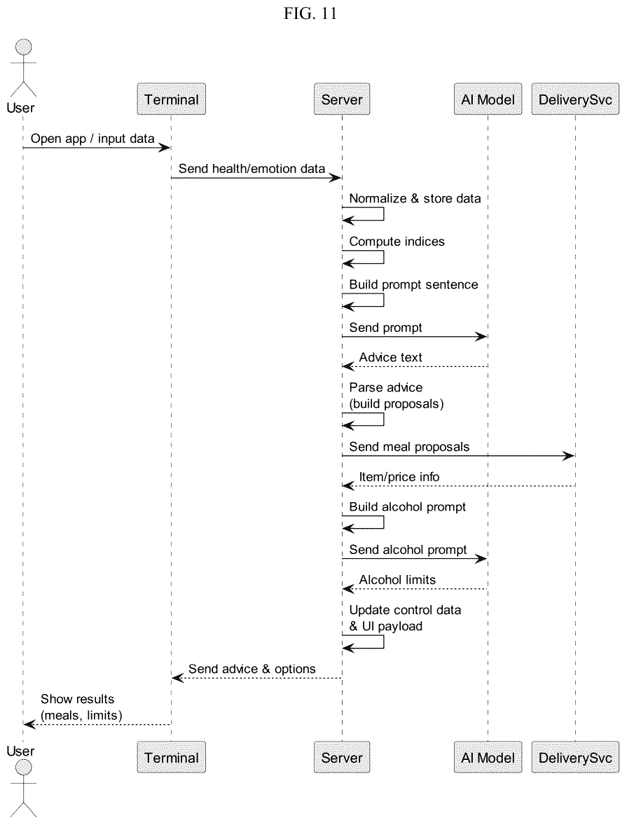

The following describes the processing flow using FIG. 11.

### Step 1:

The user operates the terminal to launch a web browser or a dedicated application and to open a health information input screen. The terminal displays input fields for individual health-related information and emotional-related information.

Input: No structured input data is present at the start of this step; the terminal only has interface configuration data.

Output: The output is a populated set of input fields on the display that the user can edit. The terminal uses its graphics subsystem to render text boxes, selection lists, and buttons, and prepares internal variables to store the user's entries.

### Step 2:

The user enters health-related information and emotional-related information into the input fields and confirms submission. The terminal captures the user's keystrokes and selections, updates internal variables, and, when the user presses a submit button, collects all field values into a structured internal object.

Input: The input is user-entered values such as age, sex, height, weight, past illnesses, allergies, lifestyle habits, and self-reported mood or stress level.

Output: The output is a structured data set, for example a key-value mapping in memory within the terminal, representing the health-related and emotional-related information. The terminal converts this mapping into a transmission-ready format and passes it to its communication module.

### Step 3:

The terminal transmits the structured health-related and emotional-related information to the server via a communication network. The terminal wraps the structured data into a message, assigns a destination address, and sends the message using a network protocol stack.

Input: The input is the structured data set created in Step 2.

Output: The output is a network request that reaches the server, containing the health-related and emotional-related information encoded according to the communication protocol. The terminal's network interface hardware and driver software handle segmentation, encapsulation, and transmission of the message.

### Step 4:

The server receives the network request via its communication interface and decodes the message to reconstruct the structured health-related and emotional-related information. The server verifies protocol headers, extracts the payload, and converts the encoded data into a server-side data structure suitable for further processing.

Input: The input is the received network message containing encoded health-related and emotional-related information.

Output: The output is a server-resident data object, such as a record or an associative array, that stores the user's health-related and emotional-related information in a machine-readable format.

### Step 5:

The server normalizes the received health-related and emotional-related information and stores it in a storage device. The server converts units (for example, from centimeters to meters), maps free-text disease names to standardized codes, and encodes emotional states into categorical or numerical values. The server then writes the normalized values into one or more database tables according to a predefined schema.

Input: The input is the unnormalized server-resident data object from Step 4.

Output: The output is a set of database records in the storage device containing normalized, structured health-related and emotional-related information. The normalization process constitutes data transformation in which the server applies mapping rules and unit conversions to generate consistent internal representations.

### Step 6:

The server retrieves the normalized health-related information from the storage device and performs statistical computation processing to generate indices. The server loads the relevant record for the user, extracts height, weight, age, and other parameters, and computes a body index (such as body mass index), a risk index, and a lifestyle index using arithmetic operations and rule-based evaluations.

Input: The input is the normalized health-related information stored in the database, including numeric values and coded categories.

Output: The output is an analysis result data object in the server's memory that contains at least a body index value, a risk index value or category, a lifestyle index value or category, and supporting parameters. The server obtains the output by applying mathematical formulas and rule sets to the input values.

### Step 7:

The server combines the analysis result with the emotional-related information to form a comprehensive health state representation. The server fetches the stored emotional-related information, such as stress level or mood category, and associates it with the analysis result data object. The server may compute additional combined metrics, for example by adjusting risk levels based on emotional stress indicators.

Input: The input is the analysis result from Step 6 and the normalized emotional-related information from the storage device.

Output: The output is an extended analysis result object that includes indices, risk classifications, lifestyle indicators, and emotional state descriptors. The server produces this output by merging and, where applicable, recomputing metrics that depend on both health-related and emotional-related inputs.

### Step 8:

The server generates a prompt sentence for a generative AI model based on the extended analysis result. The server uses sentence templates that specify positions for indices, categories, and emotional states. The server fills the templates with values from the extended analysis result, thereby performing string construction and concatenation operations.

Input: The input is the extended analysis result object from Step 7, including body index, risk index, lifestyle index, and emotional-related information.

Output: The output is a textual prompt sentence in natural language that describes the user's health status, risk level, lifestyle, and emotional state, and that requests specific types of advice. The generation process transforms structured numerical and categorical data into a formatted natural language sequence.

### Step 9:

The server transmits the generated prompt sentence to a generative AI model provided by an external system. The server constructs a request that contains the prompt sentence and one or more control parameters such as maximum output length or randomness settings. The server sends this request through its communication interface to an AI inference endpoint.

Input: The input is the prompt sentence generated in Step 8 and configuration values that specify how the generative AI model should respond.

Output: The output is a submitted response generation request that reaches the generative AI model, encoded in a format specified by the external system's interface. The server converts the textual prompt and parameter values into a serialized request body for transmission.

### Step 10:

The server receives a natural language response generated by the generative AI model and reconstructs the response text from the received data. The external system executes internal neural network computations and returns an encoded output sequence. The server decodes this sequence into a natural language string.

Input: The input is the network response containing encoded output tokens or text provided by the generative AI model.

Output: The output is a response text in natural language stored in the server's memory. The response text includes segments that describe recommended meals, ingestion substances, physical activities, stress management actions, and alcohol intake restrictions.

### Step 11:

The server parses the natural language response and extracts proposals in specific categories. The server analyzes the response text using pattern matching, keyword detection, or section boundary cues to identify sentences or paragraphs corresponding to meal proposals, ingestion substance proposals, physical activity proposals, stress management proposals, and alcohol intake restriction proposals. The server assigns each identified text segment to a category and stores them as structured items.

Input: The input is the natural language response text from Step 10.

Output: The output is a structured proposal set consisting of categorized items, each item containing a proposal type and associated textual content or derived parameters. The server derives this output by transforming unstructured natural language into discrete, labeled data elements.

### Step 12:

The server converts the structured proposal set into display information suitable for the terminal. The server groups proposals by category, adds headings or labels, and organizes them into sections. The server formats the proposals as a hierarchical data structure that can be rendered as headings, bullet lists, and descriptive text on the terminal.

Input: The input is the structured proposal set created in Step 11.

Output: The output is display information, such as a structured document description or layout-ready data, that specifies how each proposal should be presented to the user. The server's formatting operations add structure and presentation metadata to the proposal content.

### Step 13:

The server generates cooperation information for an external goods delivery service based on the meal proposals. The server scans the meal proposals for food item names and matches them to product entries in a product catalog or an interface specification for the external service. The server constructs an order candidate list with product identifiers, quantities, and optional constraints.

Input: The input is the subset of the structured proposal set corresponding to meal proposals and any available mapping information between food names and product identifiers.

Output: The output is cooperation information, comprising an order candidate list and associated parameters that can be sent to the external goods delivery service. The server obtains this output by mapping natural language food descriptions to machine-actionable order data.

### Step 14:

The server transmits the cooperation information to the external goods delivery service and receives availability and pricing information. The server sends a service-specific request containing product identifiers and quantities, and the external service returns responses indicating stock status, prices, and delivery options. The server stores these responses and integrates them into the display information.

Input: The input is the cooperation information generated in Step 13.

Output: The output is an updated display information object that embeds order options, including available items, prices, and delivery choices, alongside the meal proposals. The server derives this output by merging external service data with previously formatted advisory content.

### Step 15:

The server generates a further prompt sentence for a generative AI model to adjust alcohol intake restrictions based on the extended analysis result and the current control data. The server retrieves the current alcohol intake restriction limits from the storage device, combines them with recent health-related and emotional-related information, and constructs a new prompt that requests updated restriction recommendations.

Input: The input is the extended analysis result from Step 7 and current control data representing existing alcohol intake restrictions.

Output: The output is a further prompt sentence that describes the user's health status, emotional state, and current alcohol intake limits and requests evaluation and possible adjustment. The server creates this output by inserting current indices and control parameters into a specialized template designed for alcohol restriction management.

### Step 16:

The server transmits the further prompt sentence to the generative AI model and receives an updated natural language recommendation regarding alcohol intake. The server forms a second response generation request, sends it to the AI endpoint, and later decodes the resulting response text.

Input: The input is the further prompt sentence from Step 15 and associated generation parameters.

Output: The output is a second natural language response text that contains specific recommendations on maintaining, relaxing, or strengthening alcohol intake limits, expressed in qualitative and quantitative terms.

### Step 17:

The server extracts updated alcohol intake restriction parameters from the second natural language response and updates the control data in the storage device. The server identifies numerical limits such as maximum drinks per day and per week, parses them from the textual response, and writes these values into a control data record linked to the user.

Input: The input is the second natural language response text from Step 16 and the existing control data structure for alcohol intake restrictions.

Output: The output is an updated control data record that stores new numerical limits and related flags for alcohol consumption. The server performs data extraction, numeric conversion, and record updating operations to transform descriptive text into machine-enforceable constraints.

### Step 18:

The server sends the display information, including proposals and updated control-related messages, to the terminal. The server packages the advisory content, order options, and any messages relating to alcohol intake restrictions into a response message and transmits it through the communication network.

Input: The input is the display information prepared in Steps 12 and 14 and any newly generated explanatory text related to updated control data from Step 17.

Output: The output is a network response that the terminal receives and that contains all user-facing content for the current interaction. The server converts structured display data into a serialized format suitable for transmission.

### Step 19:

The terminal receives the network response, decodes the display information, and renders it on the display. The terminal's processor parses headings, sections, and order option elements, and uses its graphics subsystem to draw text, buttons, and selection controls. The terminal organizes the content so that analysis results, recommendations, and order options are clearly visible.

Input: The input is the network response from Step 18 containing encoded display information.

Output: The output is a visually rendered user interface on the terminal's display, presenting health indices, personalized advice, selectable meal orders, and messages related to alcohol intake restrictions. The terminal's operations convert serialized display data into on-screen graphical elements.

### Step 20:

The user reviews the information displayed on the terminal and optionally selects order options or acknowledges recommendations. The terminal captures any user interactions, such as selecting proposed meals for order or accepting lifestyle suggestions, and prepares new structured data representing these selections for possible further communication with the server.

Input: The input is the rendered interface and the user's visual perception and voluntary actions.

Output: The output is new interaction data stored in the terminal, which may include selected items and user confirmations. If the user initiates another request, this data serves as input to a new cycle beginning with data transmission to the server.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health management and incentive systems typically rely on fixed rule sets or simple scoring algorithms implemented on general-purpose computer hardware. These systems suffer from several technical limitations in how they process heterogeneous personal data, how they interface with machine learning components, and how they apply results to downstream transaction processing.

First, existing architectures generally treat health data input, artificial intelligence evaluation, and electronic payment processing as loosely coupled, sequential subsystems. Data describing an individual's medical information and emotional state is often preprocessed in ad hoc ways, and then passed to a machine learning model without a standardized, machine-interpretable and human-interpretable representation. This fragmented design leads to duplicated data transformations, inconsistent feature extraction, and increased latency, thus degrading overall computer system performance and reliability.

Second, in many existing implementations, generative artificial intelligence models are invoked as opaque “black boxes” that return free-form text. The absence of a structured prompt design and output specification causes costly post-processing on the server side, including complex parsing and error handling. This raises processor load, increases memory consumption, and introduces failure modes in which the output cannot be deterministically mapped to health evaluation states or benefit parameters. As a result, the computing system cannot robustly or efficiently integrate AI-derived health assessments into transaction-level processes such as electronic payment discount calculation.

Third, known systems often do not integrate the generation of health-related proposals (for diet, medication, stress management, and alcohol intake restriction) with real-time electronic payment incentives in a unified computational workflow. Health evaluations and recommendations may be generated offline or in separate applications, and are not natively coupled to benefit generation for electronic payment processing. This separation prevents the computing environment from optimizing data paths, reusing intermediate representations, or coordinating the timing of AI inference with discount computation, thereby causing unnecessary network communication, redundant computation, and delays in user interaction.

Fourth, prior systems lack a systematic mechanism by which a server processor constructs prompt sentences that consistently enumerate user attributes and explicitly specify required output formats for the generative AI model. Without such a mechanism, the model's behavior varies, leading to nondeterministic outputs and making it difficult for the processor to perform dependable downstream tasks, such as mapping health states to discount rates or triggering external service cooperation for ordering and restriction adjustment. This undermines the predictability and robustness of the overall computer system.

Thus, there is a need for an improved computer-implemented system and server-side processing architecture that: (i) unifies acquisition of personal medical and emotional data, (ii) performs standardized preprocessing and prompt generation for a generative AI model, (iii) obtains structured health evaluation results and benefit information suitable for automatic discount computation, and (iv) tightly integrates these AI results with electronic payment processing and external service cooperation. Addressing these issues at the architectural and processing level improves the technical functioning of the computer system itself, including data processing efficiency, robustness of AI integration, and responsiveness of electronic transaction handling.

The specific processing by the specific processing unit 290 of the data processing device 12 in Application Example 1 is realized by the following means.

The present invention provides a server comprising a processor configured to receive, from a communication terminal, input information including medical information of an individual and an emotional state of the individual; preprocess the input information to generate structured feature data and a prompt sentence describing the medical information and the emotional state; input at least one of the prompt sentence and the structured feature data into a generative artificial intelligence model to cause the generative artificial intelligence model to generate a health evaluation result that classifies a health condition of the individual into a plurality of stages and to generate benefit information based on the health evaluation result; generate, based on the health evaluation result and the benefit information, payment benefit data including at least one of a discount rate and an economic privilege to be applied in an electronic payment process; control transmission of the payment benefit data to at least one of an external payment processing device and the communication terminal; generate display data including the health evaluation result and the benefit information; control transmission of the display data to the communication terminal to cause the communication terminal to present, to the individual, proposals and benefits corresponding to the health evaluation result and the benefit information; and generate, based on the medical information and the emotional state, a further prompt sentence including instruction content relating to at least one of dietary proposals, medication proposals, stress management proposals, and adjustment of alcohol intake restriction, input the further prompt sentence into the generative artificial intelligence model to cause the generative artificial intelligence model to generate the proposals, and cooperate with an external service providing device to execute at least one of order processing and restriction adjustment processing based on the proposals. This enables a tightly integrated, computer-implemented workflow in which the server-side processor standardizes data preprocessing and prompt construction, invokes a generative AI model under explicit output constraints, obtains machine-usable health evaluation and benefit parameters, and directly applies those parameters within electronic payment and external service processing, thereby improving overall system efficiency, reliability, and responsiveness in handling health-related data and transaction incentives.

The term “processor” refers to a hardware computation unit or a combination of hardware computation units, including one or more central processing units or dedicated processing circuits, configured to execute instructions for processing data and controlling operations of the system.

The term “communication terminal” refers to an electronic device operated by an individual, including but not limited to a mobile device, a portable information terminal, or a personal computing apparatus, which is configured to transmit data to and receive data from the server via a communication network.

The term “medical information” refers to data related to the physical or mental health status of an individual, including at least age, biological sex, body metrics, medical history, allergy information, genetic predispositions, and lifestyle habits, which are used for health evaluation.

The term “emotional state” refers to information representing a psychological condition of an individual at a given time, including at least stress level, mood, or other affective indicators, which are used in conjunction with medical information for analysis and evaluation.

The term “input information” refers to a set of data received from the communication terminal, including at least the medical information of an individual and the emotional state of the individual, and optionally including other contextual attributes used for processing by the processor.

The term “preprocess” refers to a series of data processing operations performed by the processor on raw input information, including at least validation, normalization, encoding, structuring, and transformation into a format suitable for machine learning or further analysis.

The term “structured feature data” refers to a representation of the input information after preprocessing, in which the information is encoded into a predefined data structure, such as vectors, matrices, or tensors, that can be directly input into a machine learning model.

The term “prompt sentence” refers to a natural language expression or a sequence of tokens generated by the processor, which describes at least the medical information and the emotional state of an individual and specifies one or more tasks or output formats to be executed by a generative artificial intelligence model.

The term “generative artificial intelligence model” refers to a machine learning model capable of generating outputs, including at least natural language text or structured data, in response to input data or prompt sentences, and implemented using one or more learning algorithms or neural network architectures.

The term “health evaluation result” refers to information generated by the generative artificial intelligence model or derived from its output, which classifies a health condition of an individual into a plurality of stages or categories and optionally includes explanatory or confidence information.

The term “benefit information” refers to information generated based on the health evaluation result, including at least parameters or descriptions of economic privileges, discounts, incentives, or service offerings associated with the health condition of an individual.

The term “payment benefit data” refers to data generated by the processor for use in an electronic payment process, including at least one of a discount rate, a privilege identifier, a validity period, or other parameters that define an economic benefit applied to a transaction. The term “discount rate” refers to a numerical value representing a proportion or percentage by which a payment amount is reduced in an electronic payment process based on the benefit information.

The term “economic privilege” refers to a financial or service-related advantage provided to an individual, including but not limited to price reductions, bonus points, cash-back rewards, or preferential service terms, which is applied in association with a transaction or service usage.

The term “electronic payment process” refers to a sequence of operations executed using digital communication and computing resources to perform a monetary transaction, including at least receiving a payment request, calculating a payment amount, applying any applicable discount, and completing settlement.

The term “external payment processing device” refers to a computing apparatus or service separate from the server, which is configured to perform at least part of the electronic payment process, including authorization, settlement, and record keeping.

The term “display data” refers to data generated by the processor and transmitted to the communication terminal, including at least the health evaluation result and the benefit information, and formatted in a manner suitable for visual or auditory presentation to an individual.

The term “proposals” refers to recommendation information generated based on the medical information and the emotional state of an individual, including at least dietary proposals, medication proposals, stress management proposals, and adjustment of alcohol intake restriction.

The term “dietary proposals” refers to recommendations relating to the type, amount, or timing of food intake for an individual, generated based on health-related information and intended to support prevention or improvement of health conditions.

The term “medication proposals” refers to recommendations relating to use of medicinal products, including dosage, timing, or type of medication, generated in association with health-related information and intended to support preventive or therapeutic actions.

The term “stress management proposals” refers to recommendations relating to behavior, activities, or interventions intended to reduce or manage psychological stress, generated based on medical information and emotional state.

The term “adjustment of alcohol intake restriction” refers to modification of constraints or limits on consumption of alcoholic beverages for an individual, determined based on medical information and emotional state and expressed as a recommendation or control parameter.

The term “external service providing device” refers to a computing apparatus or service, separate from the server, that provides goods or services to an individual, including at least delivery services, health-related services, or other consumable offerings, and which can receive instructions from the server.

The term “order processing” refers to a sequence of operations carried out by the server and/or an external service providing device to place, modify, or confirm an order for goods or services in response to proposals or benefit information.

The term “restriction adjustment processing” refers to a sequence of operations executed by the server and/or an external service providing device to modify, enforce, or relax limitations related to behavioral constraints, including at least alcohol intake restriction.

The term “natural language sentence” refers to a sequence of words or symbols in a human language that is interpretable by human users and processable by a generative artificial intelligence model as input.

The term “description part” refers to a portion of a prompt sentence that enumerates, item by item, attributes of an individual, including at least medical information and emotional state, in a structured or semi-structured natural language format.

The term “instruction part” refers to a portion of a prompt sentence that specifies desired tasks, output types, or output formats to be produced by the generative artificial intelligence model, including at least the health evaluation result, the benefit information, and the proposals.

The term “output format” refers to a specification within the instruction part that defines the structure, type, or encoding of data to be generated by the generative artificial intelligence model, including but not limited to plain text, lists, or structured data representations.

In one embodiment, a server, a terminal, and a network-based generative AI model cooperate to implement the claimed system. The server includes at least one processor, a main memory, a non-volatile storage device, and a network interface. The processor executes control logic stored in the non-volatile storage and loaded into the main memory. The terminal includes at least one processor, a display, a touch-sensitive input device, a local storage unit such as a flash memory, and a wireless communication interface. The server and the terminal communicate via a communication network, which may include a wireless access network and a packet-switched backbone network.

The server executes server-side software implemented on a general-purpose operating system, such as a server-oriented operating system, together with an application framework implementing a web application or an application programming interface. The server software includes a data reception module, a preprocessing module, a prompt generation module, a generative AI model interface module, a health evaluation module, a benefit generation module, a payment integration module, and a display data generation module. The server further includes a data storage component implemented by a relational database system or a key-value store. The database stores structured records for user profiles, medical information, emotional state logs, health evaluation results, generated benefits, and electronic payment transactions.

The terminal executes application software implemented as a native mobile application, a web application running in a browser, or a hybrid application. The terminal application presents graphical user interface components for entry of medical information and emotional state values, for display of health evaluation results and benefits, and for interaction with electronic payment services. The terminal stores temporary data, such as cached medical profiles and benefit identifiers, in a local storage component such as a local database or preference store.

The server receives, from the terminal, input information including medical information of a user and an emotional state of the user. The terminal acquires the medical information and the emotional state by presenting a sequence of input fields and selection controls on the display.

The user operates the terminal by touching the display to enter age, biological sex, body weight, body height, information about existing medical conditions, information about allergies, lifestyle patterns such as exercise frequency or alcohol consumption habits, and self-reported emotional state, such as stress level, mood classification, or fatigue level. The terminal performs local validation, such as range checking and format checking, and then transmits the validated information to the server using a structured message over a secure communication protocol.

The server preprocesses the received input information to generate structured feature data.

The server performs a set of data normalization and encoding operations. The server converts categorical values, such as sex, presence or absence of specific diseases, lifestyle categories, and discrete emotional state labels, into numerical indicator vectors using one-hot encoding or embedding indices. The server scales continuous values, such as age, weight, and height, using normalization based on pre-stored mean and variance parameters, to reduce numerical variance and improve stability of neural network operations. The server handles missing values by applying predetermined imputation rules or by inserting special indicator values that signal missingness to the model. These preprocessing steps produce a fixed-dimension numerical feature vector representing the combined medical information and emotional state of the user. The server then converts this feature vector into a tensor representation suitable for processing by a neural network model.

In addition to structured feature data, the server generates a prompt sentence in natural language that describes the user's condition and specifies desired outputs. The prompt generation module constructs the prompt sentence using a description part and an instruction part. The description part enumerates medical attributes and emotional attributes in a consistent, itemized manner. For example, the description part may take the form:
“User profile:
Age: 30
Sex: male
Weight: 70 kilograms
Height: 175 centimeters
Past medical history: none
Allergies: none
Lifestyle: desk work, exercises twice a week
Current emotional state: slightly stressed but generally positive.”

The instruction part specifies evaluation and output requirements for the generative AI model. For example, the instruction part may be:
“Based on the above medical information and emotional state, evaluate the user's health status as one of ‘Good’, ‘Caution’, or ‘Needs Improvement’. Then recommend a discount rate between 0% and 10% to be applied to an electronic payment as an incentive for healthy behavior. Output the result as:
health status: one of ‘Good’, ‘Caution’, ‘Needs Improvement’
discount rate: numeric value between 0.00 and 0.10
reason: brief explanation.”

The server concatenates the description part and the instruction part into a single prompt sentence. For example:
“The user's age is 30, sex is male, weight is 70 kilograms, height is 175 centimeters, with no past medical history or allergies. The lifestyle is desk work, with exercise twice a week. The current emotional state is slightly stressed but generally positive. Based on this medical information and emotional state, evaluate the user's health status as one of ‘Good’, ‘Caution’, or ‘Needs Improvement’, and recommend a discount rate between 0% and 10% to be applied to an electronic payment as an incentive for healthy behavior. Output the result including: health_status, discount_rate, and a brief reason.”

The server inputs at least one of the structured feature data and the prompt sentence into a generative AI model. In one embodiment, the generative AI model is implemented as a neural network with an encoder-decoder architecture or a transformer-based architecture trained to generate structured text outputs in response to natural language prompts. The model parameters are stored in the storage device and loaded into a model execution engine running on the server processor or on a connected accelerator such as a graphics-oriented processor.

The generative AI model is trained, prior to deployment, using supervised learning on a dataset of examples comprising medical information, emotional state data, and labeled outputs that include health status categories and recommended benefit parameters. The training process uses a loss function that combines cross-entropy loss for categorical health status prediction and regression loss for discount rate prediction. The model parameters are updated using an iterative optimization algorithm that adjusts weight values to minimize the training loss over many epochs. Data augmentation techniques and regularization may be applied to improve generalization and reduce overfitting.

In one variation, the server uses two components: a discriminative neural network that consumes the structured feature data for health state classification, and a generative language model that consumes the prompt sentence for explanatory reasoning and formatting. The server combines outputs from these components to obtain both precise numerical classifications and human-readable explanations. In another variation, the server uses a single generative model that internally maps tokenized feature descriptions to token sequences representing the structured output.

The server health evaluation module interprets the outputs of the generative AI model. When the model outputs a text string describing the health status and discount rate, the server applies a deterministic parser that extracts the health status token and the numerical discount value according to the output format specified in the instruction part of the prompt. When the model provides probabilities for each health category, the server selects the category with the highest probability. The server optionally sets threshold values to avoid ambiguous classifications and may trigger a fallback procedure in case the model outputs an invalid or incomplete format. These mechanisms ensure that the health evaluation result and benefit information are always converted into machine-usable data types suitable for subsequent computation.

The server generates benefit information based on the health evaluation result. The benefit generation module applies mapping rules or a learned function that connects specific health status categories and confidence levels to ranges of discount rates or other economic privileges. For example, the server can map “Good” to a higher discount rate, “Caution” to a moderate discount rate and additional recommendations, and “Needs Improvement” to a low or zero discount rate but richer non-financial guidance. The server stores the generated benefit information in the database associated with a unique benefit identifier and a validity period.

The server generates payment benefit data configured for an electronic payment process. The payment integration module combines the discount rate, benefit identifier, validity period, and relevant user and transaction context into a structured record that can be consumed by an external payment processing device. The server maintains internal data structures that link benefits to transaction records, so that the same benefit is not applied more than once except under defined conditions. The server transmits the payment benefit data to the terminal or to an external payment processing environment over the network.

The server generates display data for the terminal. The display data includes at least the health evaluation result, such as “Good”, “Caution”, or “Needs Improvement”, and the associated benefit information, such as “5 percent discount on next electronic payment, valid for 30 days”. The display data may also include the reason or rationale text generated by the generative AI model. The server formats this display data into a structure that can be rendered by the terminal's user interface framework. The display data generation module minimizes data volume by transmitting only the fields needed for user presentation, thereby reducing network bandwidth usage and latency.

The terminal receives the display data and updates the user interface. The terminal presents the health evaluation result and the benefits in a structured layout, including labels, icons, and explanatory text. The user reviews the health status and understands how the discount or benefit is determined. In addition, the terminal may present interactive controls that allow the user to initiate electronic payments to which the benefits can be applied.

In one embodiment, the server further generates a prompt sentence focusing on proposals related to diet, medication, stress management, and adjustment of alcohol intake restriction. For example, the server constructs:
“Based on the user's age, sex, weight, height, past medical history, allergies, lifestyle, and current emotional state described above, generate: (1) dietary proposals including recommended types of food and meal timing, (2) medication proposals including general guidance consistent with preventive medicine, (3) stress management proposals including lifestyle adjustments, and (4) a recommendation on adjustment of alcohol intake restriction. Provide concise, actionable proposals.”

The server sends this prompt sentence to the generative AI model to obtain detailed proposal text. The server then constructs instructions for an external service providing device, such as a service that offers delivery of consumables or a management service that enforces alcohol intake restrictions. For example, the server converts a dietary proposal into a set of orderable items, or converts an alcohol restriction adjustment into a set of parameters such as maximum daily volume, hours during which consumption is prohibited, or notifications to be issued by the terminal.

The server cooperates with the external service providing device by transmitting appropriate control messages. For delivery services, the server may send order information including selected items, quantities, and delivery address. For restriction control services, the server may transmit parameters that cause another device or service to limit access to certain items or to send alerts to the user when limits are exceeded. These interactions demonstrate physical or operational impact beyond mere calculation or recommendation.

The system improves computer technology by optimizing the integration between data acquisition, AI-based evaluation, and transaction processing. The use of structured feature vectors and carefully designed prompt sentences standardizes the interface between the server and the generative AI model. This standardization reduces the complexity of parsing and validation of AI outputs, thereby lowering processor load and memory usage. Specifically, because the instruction part of the prompt explicitly defines the output format, the server can perform simple, deterministic parsing instead of resource-intensive natural language understanding. This leads to improved processing speed and reduced error rates when converting AI outputs into numerical discount rates and categorical health statuses.

The neural network architecture is tailored to the mixed type of data processed by the system. For example, the server may employ a multi-layer feedforward network with separate sub-networks for numerical and categorical features, merged in a later layer, or may employ an attention-based architecture that weighs contributing features differently for different outputs. The training process uses well-defined loss functions and optimization procedures, such as gradient-based updates with regularization, to produce a model that can generalize from training data to previously unseen users. The server can update the model periodically using new anonymized data to improve prediction accuracy and maintain performance under shifting user populations.

The system's design differs from a simple automation of human work because it introduces non-trivial data transformation, prompt structuring, and model integration techniques that would be difficult to perform mentally or manually. For example, the encoding of heterogeneous medical and emotional data into normalized tensors, the loop that refines prompt structure to stabilize AI outputs, and the use of explicit output format specifications enable the server to achieve consistent, low-latency, and high-throughput processing of large numbers of simultaneous evaluation and payment operations. The system also implements rule sets and fallback procedures to detect and correct anomalous AI outputs, which enhances robustness of the computer system as a whole.

The integration of the health evaluation pipeline with the electronic payment pipeline yields further technical effects. By storing benefits and discount parameters in a consistent data schema and tying them to transaction identifiers, the server can perform discount application on the payment amount using simple arithmetic operations at the point of sale, thereby avoiding repeated recomputation of AI inferences during payment operations. This separation of inference time and application time reduces the computational burden on the payment path and improves transaction latency. Network communication is also reduced because the terminal only needs to transmit compact benefit identifiers during payment, while the server looks up corresponding discount parameters locally.

Alternative embodiments may vary in the placement of the generative AI model and in the detail of the neural architecture, as long as the processor of the server performs the structured preprocessing, prompt generation, and integration steps described above. For example, the generative AI model may reside on an external AI service platform, with the server acting as a client that constructs and transmits prompt sentences and receives generated outputs. In another variation, the terminal may locally cache a lightweight model capable of performing preliminary health evaluation, while the server model performs more detailed evaluation and benefit generation. The system may also employ different generative model architectures, such as recurrent neural networks or hybrid models combining rule-based components with neural networks, provided that the server defines clear input and output formats and applies the outputs to electronic payment and external service control in the manner described.

Through these configurations, the server, the terminal, and the generative AI model cooperate in a specific and non-conventional way to process personal medical and emotional data, generate structured health evaluations and benefits, and directly control real-world processes such as application of discounts in electronic payments and cooperation with external service providing devices. This structure improves the functioning of the computer system by enhancing data management, and by enabling efficient, accurate, and robust execution of health-based benefit application workflows that would be technically difficult to achieve with conventional, loosely coupled systems.

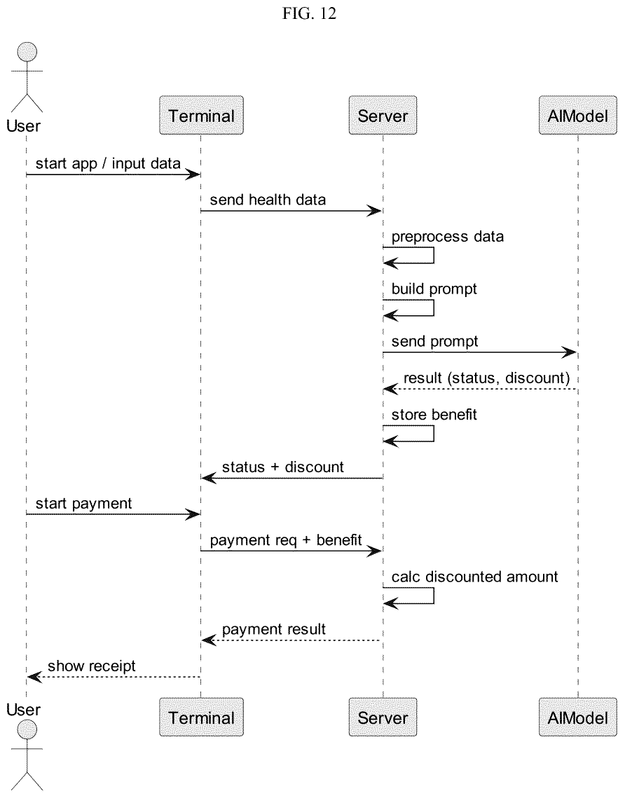

The following describes the processing flow using FIG. 12.

### Step 1:

The user operates the terminal to start a health evaluation application and open an input screen.

The terminal displays input fields for medical information and emotional state on a touch screen.

The input of this step is the user's actions (screen taps and text entry), and the output is temporary data objects in the terminal memory holding raw values such as age, sex, weight, height, past medical history, allergies, lifestyle, and emotional state.

The terminal converts each user input event into internal variables and stores them in local data structures, such as key-value pairs, while continuously updating the on-screen form.

### Step 2:

The terminal validates the entered medical information and emotional state.

The input of this step is the raw medical and emotional data stored in the local data structures of the terminal; the output is a validated data set or error messages.

The terminal performs checks such as confirming that age, weight, and height are numeric and within predefined ranges, that required fields are not empty, and that enumerated values (for example, sex or emotional state) correspond to permitted options.

The terminal generates validation flags and, in the case of invalid input, constructs and displays specific error messages, prompting the user to correct the data.

### Step 3:

The terminal packages the validated information and sends it to the server.

The input of this step is the validated data set; the output is a structured message transmitted over a network, such as a JSON-like object carried in an HTTPS request.

The terminal converts the internal variables into a structured request payload, attaches a user identifier and authentication token, and invokes an HTTP client component to send the request to a server endpoint.

The terminal sets request headers and encodes the payload, then waits for a server response while indicating progress in the user interface.

### Step 4:

The server receives and parses the input information from the terminal.

The input of this step is the structured request message; the output is an internal representation of the medical information and emotional state, stored in server memory.

The server's network interface passes the request to an application framework, which decodes the message body and maps fields such as age, sex, weight, and emotional state into typed variables or data objects.

The server performs server-side validation, logs the request, and optionally stores the raw input in a persistent storage system for later reference.

### Step 5:

The server preprocesses the input information to generate structured feature data.

The input of this step is the parsed medical information and emotional state; the output is a normalized feature vector or tensor suitable for machine learning.

The server converts categorical fields (for example, sex, disease categories, lifestyle classes, emotional labels) into numerical indicator vectors, scales numerical fields (for example, age, weight, height) using stored normalization parameters, and handles missing values by inserting default values or special “unknown” markers.

The server concatenates these encoded components into a fixed-length feature vector, then reshapes the vector into a tensor object that can be directly consumed by a neural network model.

### Step 6:

The server generates a prompt sentence that describes the user's state and specifies required outputs.

The input of this step is the parsed medical and emotional data; the output is a natural language prompt sentence.

The server constructs a description part that lists items such as age, sex, weight, height, past medical history, allergies, lifestyle, and emotional state in a consistent format.

The server appends an instruction part that defines the health evaluation task and the output format. For example, the server generates a prompt sentence such as:
“The user's age is 30, sex is male, weight is 70 kilograms, height is 175 centimeters, with no past medical history or allergies. The lifestyle is desk work, with exercise twice a week. The current emotional state is slightly stressed but generally positive. Based on this medical information and emotional state, evaluate the user's health status as one of ‘Good’, ‘Caution’, or ‘Needs Improvement’, and recommend a discount rate between 0% and 10% to be applied to an electronic payment as an incentive for healthy behavior. Output the result including: health_status, discount_rate, and a brief reason.”

### Step 7:

The server inputs the feature data and/or the prompt sentence into a generative AI model.

The input of this step is the normalized feature tensor and the prompt sentence; the output is an intermediate result from the generative AI model, such as a generated text string or structured tokens indicating health status and discount rate.

The server invokes a model interface component that passes the tensor or tokenized prompt into a neural network model implemented on the server or on an external AI service.

The generative AI model performs internal computations such as matrix multiplications, non-linear activations, and attention operations on the provided inputs, and returns a response that encodes at least a health status category and a discount value.

### Step 8:

The server interprets the generative AI model output to obtain a health evaluation result and raw benefit parameters.

The input of this step is the generated text or token sequence; the output is a structured health evaluation result and initial benefit information.

The server applies a deterministic parsing routine that searches for markers such as “health_status:” and “discount_rate:” in the generated output, extracts the associated values, and converts them into internal variables or enumeration types.

The server checks that the extracted health status is one of the permitted categories and that the discount rate lies within an allowed numerical range; if necessary, the server clips the value or triggers a fallback rule to ensure consistency.

### Step 9:

The server generates benefit information based on the health evaluation result.

The input of this step is the health status and the raw discount parameter; the output is a benefit record containing finalized benefit attributes.

The server applies predetermined mapping rules or a secondary computational function to adjust the discount rate or to attach additional attributes such as validity period, usage limits, and associated service types.

The server creates a benefit identifier, stores the benefit record in a database, and links it to the user's profile so that the benefit can be referenced during later payment operations.

### Step 10:

The server generates payment benefit data to be used during an electronic payment process.

The input of this step is the benefit record and internal user identifiers; the output is a compact payment benefit data structure.

The server selects fields required for payment processing, such as the discount rate, benefit identifier, and validity window, and omits non-essential fields to reduce payload size.

The server assembles these fields into a data object that will be associated with future payment requests, and stores a relationship between the benefit identifier and the discount rate in the payment integration module.

### Step 11:

The server generates display data containing the health evaluation result and benefit information and sends it to the terminal.

The input of this step is the health evaluation result and the benefit record; the output is a formatted response message transmitted to the terminal.

The server constructs display data including textual descriptions such as “Health status: Good” and “You have received a 5 percent discount on your next electronic payment,” and may also include a brief reason generated by the generative AI model.

The server places this display data into a response body, attaches appropriate headers, and transmits the response over the network to the terminal.

### Step 12:

The terminal receives the display data and presents the health evaluation and benefits to the user.

The input of this step is the response message from the server; the output is an updated screen view on the terminal and stored local benefit metadata.

The terminal parses the response body to extract fields such as health status, discount rate, and validity period, stores the benefit identifier and discount details in a local store, and updates the graphical user interface to show the evaluation and available benefits.

The user observes the presented information, understands the current health classification, and becomes aware of the associated discount opportunities.

### Step 13:

The user initiates an electronic payment using the terminal.

The input of this step is the user's selection of a payment function on the terminal; the output is a payment initiation request inside the terminal.

The terminal retrieves locally stored benefit identifiers and checks their validity against the current time and usage status.

The terminal constructs an internal payment request that includes the intended transaction amount and attaches any applicable benefit identifier for which the validity conditions are satisfied.

### Step 14:

The terminal sends a payment request including any applicable benefit identifier to the server or an external payment processing device.

The input of this step is the internal payment request; the output is a transmitted payment message containing transaction amount, user identifier, and benefit identifier.

The terminal uses a payment interface component to encode transaction details, appends the benefit identifier, and sends the message over the network using a secure protocol.

The terminal then waits for a confirmation or rejection message and displays a progress indicator during this waiting period.

### Step 15:

The server applies the payment benefit data to the transaction and calculates a discounted amount.

The input of this step is the payment message containing the transaction amount and benefit identifier; the output is a final payable amount and updated benefit usage status.

The server looks up the benefit record corresponding to the benefit identifier, verifies that the benefit is valid and unused, retrieves the associated discount rate, and computes the discounted amount by multiplying the transaction amount by a factor that reflects the discount.

The server updates the benefit record to mark it as used, logs the transaction, and constructs a response containing the original amount, the discount applied, and the final payable amount.

### Step 16:

The terminal receives the payment result and displays the transaction outcome to the user.

The input of this step is the payment result message from the server or payment processor; the output is a final receipt or confirmation view on the terminal.

The terminal parses the received data to obtain the original amount, discount rate, discount value, and final charged amount, and updates the user interface to show these values together with a message such as “Discount applied based on your health status.”

The user reviews the transaction details and confirms that the payment has been completed with the correct discount applied.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

In conventional computer-implemented health management systems, a processor typically applies fixed rule-based logic or static recommendation tables to personal health information to generate generic advice regarding diet or medication. Such systems are limited in that they (i) do not adaptively transform heterogeneous, user-entered health information into a machine-understandable representation optimized for advanced machine learning models, (ii) do not systematically generate structured natural-language prompt sentences tailored to an individual risk profile for input to a generative information processing model, and (iii) do not provide an integrated, closed-loop mechanism that links generated recommendations with executable actions, such as ordering goods or dynamically adjusting behavioral constraints, in a technically efficient manner.

As the complexity and volume of health-related data increase, and as generative models require carefully engineered prompts to produce reliable outputs, naive architectures that simply forward raw user data to a generative model suffer from several technical problems.

First, such architectures impose unnecessary computational overhead and bandwidth usage because unstructured or redundant data are transmitted and processed without appropriate preprocessing, thereby degrading throughput and responsiveness of the system. Second, the absence of systematic feature extraction and risk-index calculation leads to unstable or inconsistent outputs from the generative model, reducing the technical reliability of the recommendations and making it difficult to adapt the system to diverse user conditions.

Third, existing systems lack a protocolized feedback path from generated content back into the system state, such that generated recommendations cannot efficiently drive downstream machine operations, including interaction with external ordering services or dynamic enforcement and adjustment of constraints (for example, alcohol intake limits) based on a user's emotional state.

Furthermore, many known health management platforms treat emotional information, such as stress or mood, merely as an auxiliary annotation, and do not integrate such information into the core computational pipeline for feature extraction, prompt construction, and recommendation adjustment. As a result, they cannot realize fine-grained, technically controlled modulation of generated outputs in accordance with temporal changes in emotional conditions, nor can they algorithmically adjust the intensity or content of recommendations (e.g., alcohol limitation or stress management guidance) in real time.

Accordingly, there is a need for an improved computer-implemented system and server-side processing architecture that: (1) transforms heterogeneous biological and emotional information into structured data, (2) performs numerical and statistical processing to compute health indices and risk indices, (3) extracts features suitable for machine learning, (4) programmatically generates prompt sentences that accurately condition a generative information processing model, (5) converts the generated proposal information into actionable outputs linked to external goods supply services, and (6) closes the loop by updating and refining proposal information based on further prompt sentences that incorporate emotional information and extracted features. Such an architecture can improve the technical performance, stability, and utility of computer-based health recommendation systems beyond what is achievable with conventional rule-based or direct-prompt approaches.

The specific processing by the specific processing unit 290 of the data processing device 12 in Example 2 is realized by the following means.

The present invention provides a server comprising a processor configured to receive, from a terminal device, personal biological information and emotional information of an individual as structured data; to perform numerical processing and statistical processing on the structured data to calculate health indices and risk indices and to extract features based on the health indices and the risk indices; to generate a first prompt sentence, based on the biological information, the emotional information, and the extracted features, for causing an external generative information processing model to generate proposals relating to at least one of nutritional intake, exercise, and medicinal use in preventive medicine, and to input the first prompt sentence into the generative information processing model to obtain proposal information; to transmit the proposal information as visual information to the terminal device to present the proposal information to the individual and to cooperate with an external goods supply service to enable ordering of food, beverages, or lifestyle-related goods based on the proposal information; and to generate a second prompt sentence, based on the emotional information and the extracted features, for adjusting at least one of an alcohol intake limit amount and an alcohol intake frequency, to input the second prompt sentence into the generative information processing model, and to update the proposal information based on an adjustment result obtained from the generative information processing model. This enables a technically improved, end-to-end health recommendation and execution platform in which heterogeneous biological and emotional information is computationally normalized, transformed into machine-learning-ready features, and utilized to construct optimized prompt sentences for a generative information processing model, thereby yielding stable, personalized proposal information that is automatically linked to external service interfaces and dynamically updated in response to changes in emotional state.

The term “personal biological information” refers to data representing physical or physiological conditions of an individual, including, for example, past illnesses, age, sex, body mass index, blood pressure, and other health-related parameters.

The term “emotional information” refers to data representing psychological or affective states of an individual, including, for example, stress level, mood, anxiety level, or self-reported emotional status at a given time.

The term “structured data” refers to information that is represented in a predefined data format or schema, such as key-value pairs, tables, or hierarchical records, which allow a processor to systematically parse, validate, and process the information.

The term “terminal device” refers to an electronic apparatus operated by an individual, such as a mobile communication device, a portable computing device, or a stationary computing device, that is capable of inputting information and communicating with a server over a communication network.

The term “numerical processing” refers to operations performed by a processor on numeric values, including, for example, arithmetic calculations, scaling, normalization, and transformation of raw input values into derived numeric indicators.

The term “statistical processing” refers to operations performed by a processor to compute statistical measures from data, including, for example, averages, variances, standard deviations, distributions, categorical statistics, and other measures used to derive health indices and risk indices.

The term “health indices” refers to quantitative or categorical indicators derived from personal biological information, which summarize aspects of health status, such as cardiovascular risk level, body mass index category, blood pressure category, or activity level.

The term “risk indices” refers to quantitative or categorical indicators representing likelihoods or severities of medical or health-related risks, calculated based on biological information, emotional information, or a combination thereof.

The term “features” refers to machine-interpretable variables extracted from input data and indices, including numeric vectors, categorical encodings, or other representations, that are suitable for input to machine learning models or other computational analysis.

The term “prompt sentence” refers to a natural-language or structured text expression generated by a processor, which describes input conditions and desired output constraints in a form suitable for conditioning an external generative information processing model.

The term “generative information processing model” refers to a computational model, such as a generative AI model, that generates output information, including text or other content, in response to an input prompt sentence, based on parameters learned from training data.

The term “proposal information” refers to information generated by the generative information processing model in response to a prompt sentence, including, for example, recommendations or guidance relating to nutritional intake, exercise, medicinal use, or other preventive healthcare activities.

The term “nutritional intake” refers to consumption-related behaviors and quantities involving food and beverages, including, for example, recommended types of nutrients, target amounts of calories, salt, fat, or other dietary components per unit time.

The term “exercise” refers to physical activities intended to improve or maintain health, including, for example, aerobic exercise, resistance training, walking, stretching, and associated frequency, duration, and intensity parameters.

The term “medicinal use” refers to utilization of pharmaceutical products or medical treatments, including, for example, recommended consultation with a medical professional, possible administration of medications, and guidance regarding dosage or timing, subject to medical oversight.

The term “visual information” refers to data formatted for display on a display unit of a terminal device, such as text, symbols, icons, or graphical elements, that convey proposal information to an individual in human-readable form.

The term “external goods supply service” refers to a service provided by an external system that supplies physical goods, including, for example, food items, beverages, or lifestyle-related goods, and that can receive and process orders via a communication interface.

The term “alcohol intake limit amount” refers to a quantitative upper bound on a volume or quantity of alcoholic beverages to be consumed by an individual over a specified period.

The term “alcohol intake frequency” refers to a prescribed or recommended number of occurrences or intervals at which an individual is permitted or advised to consume alcoholic beverages over a specified period.

The term “stress-relief behaviors” refers to activities intended to reduce mental or physical stress, including, for example, relaxation techniques, breathing exercises, light physical activity, or leisure activities.

The term “sleep habits” refers to patterns and characteristics of sleep behavior, including, for example, bedtime, wake-up time, sleep duration, and sleep-related routines.

The term “lifestyle habit improvement” refers to modifications to daily routines and behaviors, including, for example, adjustments to diet, physical activity, sleep, substance use, and work-rest balance, aimed at improving overall health.

The term “group of foods” refers to a set of food items categorized according to suitability for an individual, nutritional characteristics, or health-related criteria derived from biological and emotional information.

The term “group of beverages” refers to a set of beverage items categorized according to suitability for an individual, nutritional or compositional characteristics, or health-related criteria derived from biological and emotional information.

The term “order candidates” refers to one or more items or item combinations that are presented to an individual as potential selections for ordering from an external goods supply service based on proposal information.

In one embodiment, a server, a terminal, and a user cooperate to implement the system. The server includes at least one processor, a memory, a storage unit, and a network interface. The server operates on general-purpose computing hardware, such as a multi-core central processing unit executing an operating system, and may run on a physical machine or a virtual instance. The server executes an application program written, for example, in a high-level programming language and using a web application framework. The server further executes data-processing libraries corresponding to data-frame processing and numerical computing, as well as a machine learning framework implementing a neural-network model. The terminal includes a display unit, an input unit such as a touch panel, a network interface, and a local processor. The terminal executes an application program providing a graphical user interface for the user to enter personal biological information and emotional information and to view generated proposal information.

The server stores, in the storage unit, a program that causes the processor to receive personal biological information and emotional information of the user as structured data from the terminal. The server defines a data schema in which the structured data is represented, for example, as key-value pairs or hierarchical records. The server configures a data model such that fields for age, sex, past illnesses, current symptoms, blood pressure, body mass index, exercise habits, sleep patterns, and other health-related parameters are included as biological information, and such that stress level, mood, anxiety, irritability, and self-reported emotional states are included as emotional information. The server defines the structured data in a format such as a record that can be directly loaded into a data-frame object.

The terminal displays input screens for biological information and emotional information using a user-interface toolkit. The terminal allows the user to select items from lists or to enter numerical values and free text, such as “hypertension,” “age 45,” “male,” “blood pressure often around 150/95,” “BMI 28,” “sedentary lifestyle,” “stress level: high,” and “feeling anxious in the evening.” The terminal validates the input locally according to data types defined by the server, such as integer, floating-point, and controlled vocabulary strings.

The terminal then transmits the input information to the server according to the structured schema.

The server loads the structured data into a data-frame object using a data processing library and converts text representations into normalized numeric values. The server parses, for example, a text such as “150/95” into systolic and diastolic pressure fields and converts them into integer values. The server converts categorical values such as sex, smoking status, and exercise frequency into standardized labels or codes. The server employs numerical computing functions to calculate derived indicators, such as body mass index from weight and height, weekly exercise minutes from frequency and duration, and approximate sleep debt from bedtime and wake-up time information.

The server defines health indices, such as a cardiovascular risk index, a metabolic risk index, and a lifestyle balance index, and computes these indices from the biological information and the derived indicators using predetermined formulas and statistical operations. The server uses, for example, weighted sums, normalization functions, and standard deviation-based scaling to transform the raw data into normalized scores. The server thus reduces heterogeneous user-entered data to a compact set of machine-interpretable health indices. The server also defines risk indices, such as an alcohol-related risk index and a stress-related risk index, which are calculated from combinations of biological information, emotional information, and health indices.

The server performs feature extraction by transforming the health indices and risk indices into feature vectors suitable for input to a neural network. The server uses, for example, one-hot encoding for categorical items, min-max scaling or z-score normalization for numerical items, and concatenation of these encodings into a fixed-length feature vector. The server may additionally incorporate time-based features, such as recency of symptom updates or temporal trends of stress level, by appending exponential moving averages or differences between consecutive values.

The server executes a neural-network model implemented in a machine learning framework.

In one embodiment, the server uses a feed-forward neural network comprising an input layer corresponding to the dimensionality of the feature vector, one or more hidden layers configured with fully connected nodes and non-linear activation functions, and an output layer generating intermediate latent features. The server defines, for example, hidden layers with rectified linear unit activation and may employ techniques such as dropout or batch normalization to stabilize training and inference. In another embodiment, the server uses a transformer-based encoder network that processes sequences of features, particularly when time-series health data is available. The server stores pre-trained model parameters, such as weight matrices and bias vectors, in the storage unit.

The server trains the neural-network model prior to deployment using training data including historical biological information, emotional information, and associated medically curated labels indicating health risk categories or recommended intervention intensities. During training, the server defines a loss function, such as cross-entropy loss for classification of risk categories or mean squared error for regression of risk scores. The server performs gradient-based optimization using an algorithm such as stochastic gradient descent or an adaptive moment estimation method to update the weight parameters of the network. The server may also apply techniques such as data augmentation, regularization, and early stopping to prevent overfitting. Through the training process, the server learns non-linear mappings from heterogeneous input information to compact latent features that encode health-related risk patterns and preference patterns more efficiently than manually designed rule-based systems.

During operation, the server inputs the feature vector corresponding to the current user into the trained neural network to obtain an output latent feature vector. The server interprets elements of this latent feature vector as probabilities or intensities associated with health risk categories and behavioral intervention recommendations. The server applies thresholding or soft assignment to convert continuous outputs into descriptive labels such as “elevated cardiovascular risk,” “overweight,” “insufficient physical activity,” “high stress level,” and “sensitive to alcohol intake.” By performing such computations within the neural network, the server can identify complex, non-linear relationships that are difficult to capture with conventional rule-based processing, thus improving classification accuracy and stability of health-related inferences.

The server constructs a prompt sentence for a generative AI model based on the biological information, the emotional information, and the extracted features. The server uses a template mechanism in which predefined sentence structures include placeholders for age, sex, past illnesses, blood pressure category, BMI category, lifestyle indicators, emotional status, and latent feature-derived labels. The server inserts specific numeric values and categorical labels into the template. The server may include explicit instructions regarding output format, level of detail, and constraints related to preventive medicine. For example, the server generates a prompt sentence such as:
“User health information:
   Age: 45
   Sex: male
   Past illnesses: hypertension
   Current status: blood pressure around 150/95, BMI 28, sedentary office work, no smoking, occasional alcohol.
   Emotional status: high stress level, anxious in the evening.
Derived analysis from server-side neural network:
   Elevated cardiovascular risk
   Overweight (BMI category: overweight)
   Insufficient physical activity
   Increased sensitivity to alcohol intake

You are a generative AI model specializing in preventive medicine.

Based on the above information, generate detailed and practical recommendations for:
1. Nutrition (including specific targets such as daily salt intake, maximum alcohol intake per week, and calorie balance),
2. Exercise (frequency, duration, and intensity appropriate for an individual with hypertension and high stress),
3. Medication or medical follow-up (if appropriate), and
4. Stress management behaviors and sleep habits.

Write the answer in clear English suitable for a layperson, organized with headings for ‘Diet’, ‘Exercise’, ‘Medication/Follow-up’, and ‘Stress management’.”

The server, in another example, generates a more concise prompt sentence when only limited information is available. For instance, the server generates:

“User health information: hypertension, age 45, male, BMI 28, sedentary lifestyle, high stress level.

Act as a generative AI model specialized in preventive medicine.

Based on this data, generate concise recommendations for optimal nutrition intake, exercise, and alcohol intake limits, including numerical targets for daily salt intake, weekly exercise duration, and maximum weekly alcohol consumption.”

The server transmits the constructed prompt sentence to an external generative information processing model via a network interface. The server uses an application programming interface to send the prompt sentence as part of a request that specifies model parameters such as output length, temperature, and response format. The external model implements a transformer-based architecture with multiple self-attention layers, feed-forward layers, and layer normalization, and outputs token sequences representing natural language text proposals. The server receives the token sequence output and reconstructs the response text.

The server parses the response text according to headings or special markers included in the prompt sentence. The server separates sections such as “Diet,” “Exercise,” “Medication/Follow-up,” and “Stress management,” and assigns each section to a corresponding field in an internal representation of proposal information. The server may examine the response text for numerical values, such as “limit daily salt intake to 6 g or less” or “perform aerobic exercise at least 30 minutes, three times per week,” and verify that they fall within medically acceptable ranges defined in configuration data. The server adds disclaimers or annotations as needed to clearly indicate that the proposals are not a medical diagnosis.

The server converts the proposal information into visual information for display on the terminal. The server defines a data structure that includes headings, bullet points, explanatory paragraphs, and possibly icons or color-coded indicators corresponding to risk levels. The server transmits the visual information to the terminal using a communication protocol. The terminal receives the visual information, converts it into user-interface components, and displays the proposal information to the user on the display unit. The terminal allows the user to scroll through the content, expand or collapse sections, and mark items as “to be followed” or “completed.”

The server interacts with an external goods supply service to enable ordering of food, beverages, or lifestyle-related goods based on the proposal information. The server maintains a mapping between categories or names of foods and beverages recommended in the proposal information and product identifiers provided by the external service. When the proposal information includes a recommendation such as “increase intake of vegetables and whole grains” or “replace sugary drinks with non-alcoholic beverages,” the server identifies corresponding product candidates, such as vegetable sets, whole grain products, or non-alcoholic drinks, in the catalog of the external service. The server sends order candidate information to the terminal, including product names, descriptions, and prices. The terminal displays order candidates in association with the relevant recommendations and allows the user to select and confirm orders. The server then communicates order information to the external service through an application programming interface.

The server further uses the emotional information and the extracted features to generate a second prompt sentence for adjusting alcohol intake limits. The server defines rules or learned mappings that relate emotional states and risk indices to dynamic adjustment of alcohol intake recommendations. For example, the server increases restriction when the stress level and alcohol-related risk index exceed thresholds. The server describes the current emotional status, the health indices, and the desired adjustment logic in the second prompt sentence and sends this prompt sentence to the generative model. The generative model outputs an adjusted alcohol intake policy, expressed in natural language, which may state, for example, “limit alcohol to at most one drink on no more than two days per week” when the stress and risk indices are high, or may relax restrictions under more favorable conditions.

The server incorporates this adjustment into the proposal information and updates the displayed recommendations at the terminal.

The server generates additional prompt sentences based on the extracted features to obtain stress management proposals. The server describes derived features such as high stress index, poor sleep habits, and irregular work-rest balance and requests concrete stress-relief behaviors and sleep habit improvements. The generative model outputs detailed behaviors such as “practice deep-breathing exercises for 5 minutes before bedtime,” “avoid screen exposure 30 minutes before sleep,” or “perform light stretching during work breaks.” The server integrates these recommendations with existing nutritional and exercise guidance, so that the user receives a coherent and comprehensive plan.

The server achieves technical improvements in several respects. By converting heterogeneous biological and emotional information into structured data and then into normalized feature vectors, the server reduces the dimensionality and redundancy of input data, which decreases memory usage and improves cache locality during neural-network inference. The use of precomputed health indices and risk indices as intermediate representations allows the server to perform inference with smaller models than would be required if raw data were directly provided to the generative model, thereby reducing processing time and network bandwidth consumption. The feature extraction and neural-network inference pipeline enables more stable and accurate estimation of risk patterns compared with rule-based systems, reducing variability and errors in subsequent recommendations.

The server also improves technical performance by organizing the prompt sentences according to templates that encode best practices for conditioning generative models. This reduces the need for repeated trial-and-error prompt design at runtime and leads to more consistent and predictable response structures from the generative model. The server, by structuring both inputs and outputs and by performing additional verification of numerical recommendations, reduces inappropriate or out-of-range outputs, thereby increasing system robustness.

The system differs from mere automation of human reasoning because the server employs computational mechanisms that are not ordinarily performable by a human in real time. These mechanisms include high-dimensional vector computations, gradient-based training of neural networks, statistical normalization processes, and systematic mapping of features to generative prompts that are optimized for machine consumption. The server, by implementing a specific combination of feature extraction, neural-network inference, and prompt generation, improves the way computer resources are used to generate health-related recommendations. For example, the server can batch-process feature vectors for multiple users, reuse loaded model parameters in memory, and cache frequently used template segments, all of which reduce latency and overhead compared to naive architectures that repeatedly transmit unprocessed raw data to external models.

In alternative embodiments, the server may employ different neural-network architectures for feature extraction, such as recurrent neural networks when temporal sequences of health events are available, convolutional networks when processing signals such as heart rate variability, or graph neural networks when modeling relationships among different health indicators. The server may further use different generative models, such as encoder-decoder architectures or smaller domain-specific generative models, provided that they can process prompt sentences and output textual recommendations. The server may adapt model complexity and frequency of inference according to available hardware resources and latency requirements.

In another embodiment, the terminal performs some preprocessing of sensor data. For example, the terminal may obtain step counts or heart rate data from built-in sensors or wearables, compute short-term summaries, and send these summaries as part of the structured data. The server then integrates these additional indicators into the same feature extraction and neural-network pipeline. The terminal may also locally cache proposal information and only request incremental updates when the user's biological information or emotional information changes beyond predefined thresholds, reducing network traffic and server load.

In another embodiment, the server may be configured to operate in a secure local network, for example in a clinical environment, and to interact with internal ordering systems instead of external goods supply services. The same computational pipeline of feature extraction, prompt generation, and generative-model-based recommendation is used, but the mapping of proposal information to orderable items corresponds to internal stock or formulary databases.

Through these structures and operations, the server, the terminal, and the user collaborate in a system that is technically improved over conventional systems. The server performs machine-optimized transformations and structured prompt generation to control a generative AI model, and converts the generated content into actionable proposals and order operations linked to the external world. This integration of advanced feature extraction, specialized neural network processing, and prompt sentence design produces more accurate, efficient, and reliable health-related proposals than naive or purely rule-based approaches, thereby enabling the invention to be implemented in a concrete and technically advantageous manner.

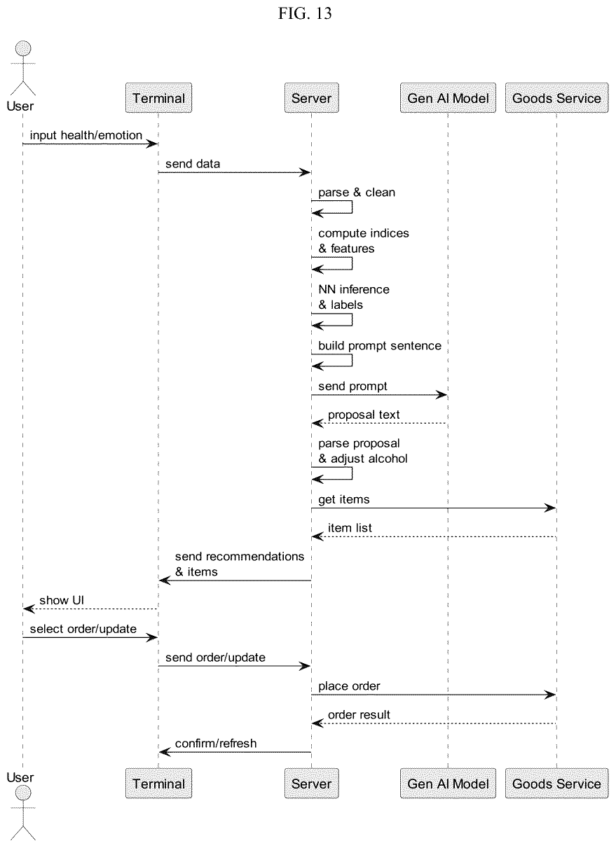

The following describes the processing flow using FIG. 13.

### Step 1:

The user operates the terminal to launch an application and input personal biological information and emotional information. The user enters, as input, items such as age, sex, past illnesses, blood pressure description, body weight and height, exercise habits, sleep habits, alcohol consumption, stress level, and current mood. The terminal validates these inputs (for example, checks that age is a number and mandatory fields are filled) and converts them into an internal structured representation. The terminal outputs validated structured data, including both biological fields and emotional fields, for transmission to the server.

### Step 2:

The terminal sends the validated structured data to the server. The terminal takes, as input, the internal structured representation created in Step 1, serializes it into a request message according to a predefined schema, and attaches metadata such as a timestamp and user identifier (pseudonymized if necessary). The terminal outputs a network request containing the structured data and transmits it through a communication interface to the server.

### Step 3:

The server receives and parses the structured data from the terminal. The server takes, as input, the network request containing the structured data, verifies its integrity, and parses the payload into an in-memory data object. The server performs basic checks, such as verifying version numbers of the schema and presence of required fields. The server outputs a normalized data object that separates biological information, emotional information, and metadata into distinct fields ready for further processing.

### Step 4:

The server converts the normalized data object into a tabular data-frame representation and performs data cleaning. The server takes, as input, the normalized data object from Step 3 and loads it into a data-frame structure. The server applies data-processing operations to parse string fields (for example, splitting “150/95” into systolic and diastolic values), convert types (for example, converting age from string to integer), and standardize categorical values (for example, mapping different representations of “male” or “female” to canonical codes). The server outputs a cleaned data-frame in which all relevant fields are correctly typed and normalized, providing a consistent basis for numerical and statistical processing.

### Step 5:

The server performs numerical computations and statistical processing to derive health indices and risk indices. The server takes, as input, the cleaned data-frame from Step 4 and applies numerical operations to calculate derived values such as body mass index from weight and height, weekly exercise minutes from frequency and duration fields, and approximate sleep duration from bedtime and wake-up time. The server also applies statistical normalization, such as z-score or min-max scaling, to map values onto standardized ranges and computes composite health indices (for example, cardiovascular risk index, metabolic risk index) by combining multiple normalized values using weighted sums and non-linear functions. The server outputs a set of health indices and risk indices appended as additional columns to the data-frame.

### Step 6:

The server extracts machine-learning features from the health indices, risk indices, and selected raw fields. The server takes, as input, the augmented data-frame from Step 5 and encodes categorical variables using techniques such as one-hot encoding, converts boolean fields into numeric flags, and applies scaling to all numerical fields to fit within ranges appropriate for neural-network input. The server concatenates these encoded values into a fixed-length feature vector representing the user's biological and emotional state as well as computed indices. The server outputs a numerical feature vector suitable for direct input to a neural-network model.

### Step 7:

The server performs neural-network inference to derive latent features and risk pattern estimates. The server takes, as input, the feature vector from Step 6 and feeds it into a pre-trained neural-network model implementing, for example, a multi-layer perceptron or transformer-based encoder. The server executes matrix multiplications, bias additions, and non-linear activation functions across the network layers, optionally using regularization layers such as dropout or normalization layers. The server interprets the model outputs as probabilities or scores for categories such as “elevated cardiovascular risk,” “insufficient physical activity,” “overweight,” “high stress,” and “alcohol sensitivity.” The server outputs a latent feature vector together with derived labels or scores that summarize the user's risk patterns and health-related characteristics.

### Step 8:

The server generates descriptive labels and thresholds from the neural-network outputs. The server takes, as input, the latent feature vector and associated scores from Step 7 and applies thresholding or ranking rules to determine which conditions should be flagged as significant. For example, the server compares a cardiovascular risk score to a configured threshold and decides whether to assign the label “elevated cardiovascular risk.” The server also maps continuous stress and alcohol sensitivity scores to discrete categories such as “low,” “moderate,” or “high.” The server outputs a set of human-readable labels and categories that can be embedded into a prompt sentence and used to structure subsequent recommendations.

### Step 9:

The server constructs a primary prompt sentence for a generative AI model based on biological information, emotional information, and extracted labels. The server takes, as input, the original cleaned data, the health indices, the risk indices, and the descriptive labels from Step 8. The server fills a textual template that contains placeholders for age, sex, past illnesses, current health status, emotional status, and derived analysis. The server inserts specific numeric values (for example, blood pressure, BMI, exercise minutes) and qualitative labels (for example, “elevated cardiovascular risk,” “high stress level”). The server also appends explicit instructions to request proposals on nutrition, exercise, medication, and stress management. The server outputs a complete prompt sentence in natural language, such as:
“User health information:
   Age: 45
   Sex: male
   Past illnesses: hypertension
   Current status: blood pressure around 150/95, BMI 28, sedentary office work, no smoking, occasional alcohol.
   Emotional status: high stress level, anxious in the evening.
Derived analysis from server-side neural network:
   Elevated cardiovascular risk
   Overweight (BMI category: overweight)
   Insufficient physical activity
   Increased sensitivity to alcohol intake
   You are a generative AI model specializing in preventive medicine.

Based on the above information, generate detailed and practical recommendations for:
1. Nutrition (including specific targets such as daily salt intake, maximum alcohol intake per week, and calorie balance),
2. Exercise (frequency, duration, and intensity appropriate for an individual with hypertension and high stress),
3. Medication or medical follow-up (if appropriate), and
4. Stress management behaviors and sleep habits.

Write the answer in clear English suitable for a layperson, organized with headings for ‘Diet’, ‘Exercise’, ‘Medication/Follow-up’, and ‘Stress management’.”

### Step 10:

The server sends the primary prompt sentence to the generative AI model and obtains proposal information. The server takes, as input, the constructed prompt sentence from Step 9 and packages it into a request for an external generative information processing model using an application programming interface. The server specifies model parameters such as response length and randomness level. The generative AI model processes the prompt internally and returns an output text composed of sections corresponding to the requested categories. The server outputs the raw generated text, which contains proposed nutritional guidance, exercise plans, medication follow-up suggestions, and stress management recommendations.

### Step 11:

The server parses and structures the proposal information returned by the generative AI model. The server takes, as input, the raw generated text from Step 10 and analyzes it according to headings or delimiters that were requested in the prompt (for example, “Diet,” “Exercise,” “Medication/Follow-up,” “Stress management”). The server splits the text into segments associated with each heading and extracts important numerical targets, such as “limit daily salt intake to 6 g or less” or “perform aerobic exercise at least 30 minutes, three times per week.” The server converts these segments into a structured internal representation with fields for each recommendation category and associated parameters. The server outputs structured proposal information suitable for both display and further processing.

### Step 12:

The server generates a secondary prompt sentence to adjust alcohol intake recommendations based on emotional and risk-related features. The server takes, as input, the emotional information, the alcohol-related risk index, and the labels from Step 8, as well as any initial alcohol-related suggestions extracted in Step 11. The server evaluates whether the stress level and alcohol risk exceed predefined thresholds, and determines if stricter limits are needed.

The server then constructs a secondary prompt sentence that summarizes the current alcohol recommendation, emotional state, and risk indices and requests the generative AI model to refine or tighten the alcohol intake guidance. The server outputs a secondary prompt sentence that explicitly focuses on adjusting at least one of alcohol intake limit amount and alcohol intake frequency.

### Step 13:

The server sends the secondary prompt sentence to the generative AI model and updates the alcohol-related part of the proposal information. The server takes, as input, the secondary prompt sentence from Step 12 and transmits it to the generative AI model via the application programming interface. The generative AI model returns refined text specifying updated alcohol limits, for example “limit alcohol to no more than one drink on at most two days per week during high-stress periods.” The server replaces or augments the alcohol-related portion of the proposal information with this updated guidance. The server outputs updated structured proposal information that includes dynamically adjusted alcohol recommendations consistent with the user's emotional state and risk indices.

### Step 14:

The server maps recommended foods and beverages in the proposal information to items in an external goods supply service. The server takes, as input, the structured proposal information from Step 13 and a catalog of products obtained from the external goods supply service. The server identifies recommended food categories (for example, “high-fiber vegetables,” “whole grains,” “non-alcoholic beverages”) in the proposal information and searches the catalog for matching or similar items by category, nutritional tags, or product metadata. The server constructs order candidate lists that link each recommendation to one or more specific products in the catalog. The server outputs a set of order candidates with product identifiers, descriptions, and prices, each associated with corresponding health recommendations.

### Step 15:

The server formats the proposal information and order candidates into visual information and sends it to the terminal. The server takes, as input, the structured proposal information and the order candidates from Step 14. The server arranges the content into a layout specification including headings, bullet points, explanatory text, and optional icons or emphasis for important warnings. The server associates each health recommendation with any related order candidates, so that the terminal can display these together. The server outputs a response message that contains the formatted visual information for rendering by the terminal.

### Step 16:

The terminal receives and renders the visual information for presentation to the user. The terminal takes, as input, the response message from Step 15, parses the layout specification, and instantiates user-interface components such as text labels, list items, buttons, and images. The terminal displays sections for “Diet,” “Exercise,” “Medication/Follow-up,” and “Stress management,” including concrete recommendations like “limit daily salt intake to 6 g or less” and “perform aerobic exercise at least 30 minutes, three times per week.” The terminal also displays associated order candidates, such as specific low-salt food products or non-alcoholic beverages, with selectable controls for ordering. The terminal outputs a graphical presentation that the user can interact with.

### Step 17:

The user reviews the recommendations and optionally selects order candidates or updates personal information. The user takes, as input, the visualized proposal information and order options on the terminal screen, evaluates them, and may tap to place orders for certain items or to mark some recommendations as “to follow.” The user may also choose to update biological or emotional information, for example by indicating that stress has decreased or that a new exercise routine has been started. The terminal captures these user actions and outputs updated structured data or order commands to the server.

### Step 18:

The server processes user feedback and initiates order operations or recalculates recommendations as needed. The server takes, as input, the user's order selections and any updated information from Step 17. For order selections, the server converts chosen order candidates into order requests and sends them to the external goods supply service through its interface. For updated personal information, the server re-enters the processing pipeline starting from data parsing and feature extraction, recomputes indices, regenerates prompt sentences, and obtains updated proposal information from the generative AI model. The server outputs confirmation messages for successful orders and refreshed recommendations when user data changes, thereby maintaining a closed-loop system that adapts to evolving biological and emotional states.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented health recommendation systems generally adopt a static, rule-based architecture in which a server retrieves health parameters from a database, applies predetermined threshold checks (for example, for blood pressure or body mass index), and outputs fixed templates of advice. Such systems suffer from several technical limitations at the level of data handling, model interaction, and user-interface integration.

First, conventional systems do not dynamically restructure heterogeneous data streams (biometric measurements, emotional indicators, user preferences, and behavioral histories) into a form optimized for interaction with a generative AI model. Instead, they typically pass raw or minimally processed values to a recommendation engine, which leads to suboptimal use of computational resources and model capacity. As a result, the server cannot efficiently calculate time-series statistics, correct outliers, and normalize values in a unified pipeline, and thus cannot provide stable and predictable inputs to a generative AI model. This degrades model performance and increases the probability of inconsistent or noisy outputs.

Second, existing systems generally treat natural-language prompts for generative AI models as static or manually curated strings. There is no integrated mechanism in the server to automatically construct prompt sentences that encode, in a machine-optimized way, complex combinations of biometric information, emotional information, preferences, and behavior history, nor to refine such prompt sentences based on feedback from users. Consequently, the interaction between the server and the generative AI model is inefficient: the same or similar prompts are repeatedly used regardless of user state, which reduces the contextual relevance of generated proposals and wastes computational cycles on the model side.

Third, conventional architectures lack a unified flow for converting generative AI outputs into structured, machine-processable data that can drive downstream computer-implemented services, such as integration with external food-ordering platforms. Without a systematic transformation of AI-generated text into structured data including nutrient information, intake restriction information, and emotion-correspondence information, the server must rely on ad-hoc parsing or manual mapping, which increases processing latency, complexity of the code base, and susceptibility to parsing errors. This prevents reliable automatic generation of order instructions for external food provision services and hinders real-time user interaction.

Fourth, known systems do not tightly couple emotional analysis with dynamic adjustment of alcohol-intake restrictions at the system level. Emotional analysis, when present, is often used only for display or logging, and does not feed back into the computational logic that generates prompts and controls the generative AI model. Therefore, the server cannot systematically alter proposal generation when detecting high stress or other emotional states, and cannot compute or enforce a restriction policy regarding alcoholic beverages in a technically consistent manner.

Fifth, typical systems lack a feedback loop that uses user evaluation information to automatically modify prompt configuration and model input conditions. Even when user feedback is collected, it is often stored as unstructured logs and not used in a structured pipeline to adjust prompt templates or input parameters. As a result, the system cannot learn from past interactions at the level of prompt construction, and the quality and efficiency of generative AI interactions remain static over time.

Accordingly, there is a need for an improved computer-implemented system in which a processor is configured to (i) acquire and preprocess heterogeneous user state information into analysis data suitable for generative AI interaction, (ii) automatically construct and refine prompt sentences in natural language for a generative AI model, (iii) convert model outputs into structured data that can be directly consumed by downstream components including external service interfaces, (iv) determine and apply alcohol-intake restriction policies based on emotional analysis by generating specialized prompt sentences, and (v) update prompt configuration and model input conditions based on user evaluation information. Such a system can improve the technical functioning of the server and the cooperation between the server and the generative AI model, thereby enabling more efficient, accurate, and adaptive computer-implemented health-management processing.

The specific processing by the specific processing unit 290 of the data processing device 12 in Application Example 2 is realized by the following means.

The present invention provides a server comprising a processor configured to acquire biometric information and emotional information of an individual and to store the biometric information and the emotional information as state information, to preprocess the state information, preference information, and behavior history information of the individual as analysis data by calculating time-series statistics, correcting outliers, and normalizing numerical values, to generate, based on the analysis data and the behavior history information, a prompt sentence in a natural language for causing a generative AI model to generate a proposal related to a meal or a drug based on preventive medicine and to cause the generative AI model to input the prompt sentence, to analyze an output proposal from the generative AI model and convert a meal proposal item in the output proposal into structured data including nutrient information, intake restriction information, and emotion-correspondence information and convert the structured data into display data to be presented to the individual, to associate the meal proposal item in the structured data with item information of an external food provision service, to generate order information for food based on the proposal, and to transmit the order information to the external food provision service via a communication network to enable ordering by the individual, to determine a restriction policy regarding intake of an alcoholic beverage based on an analysis result of the emotional information, to generate an alcohol-intake-restriction prompt sentence reflecting the restriction policy, and to cause the generative AI model to input the alcohol-intake-restriction prompt sentence to adjust the proposal, and to acquire evaluation information from the individual regarding the proposal and update at least one of a configuration of the prompt sentence and an input condition to the generative AI model based on the evaluation information. This enables the server to implement an improved technical pipeline in which heterogeneous user state data are transformed into optimized analysis data for generative AI processing, context-adaptive prompt sentences are automatically constructed and refined, AI-generated text is converted into structured data that directly drive external service integration, and emotional analysis and user feedback are fed back into prompt generation and model input control, thereby enhancing computational efficiency, stability of model interaction, and adaptability of the overall computer-implemented health-management system.

The term “biometric information” refers to information indicative of a physical or physiological state of an individual, including, for example, body weight, body height, heart rate, blood pressure, body mass index, activity amount, and sleep status, which can be measured by a sensor device or entered by the individual.

The term “emotional information” refers to information indicative of a psychological or affective state of an individual, including, for example, stress level, mood, and emotional category, which may be derived from text input, voice characteristics, facial expressions, or behavior patterns.

The term “state information” refers to data representing a current or recent condition of an individual, the data including at least biometric information and emotional information and optionally including derived indicators calculated from such information.

The term “preprocess” refers to performing data processing on raw input data to convert the raw input data into analysis data suitable for further computation, the data processing including at least one of calculating time-series statistics, correcting outliers, normalizing numerical values, encoding categorical values, and generating derived indicators.

The term “preference information” refers to data representing favored or disfavored items or conditions of an individual, including, for example, favorite cuisines, disliked ingredients, dietary style, and constraints such as vegetarianism, veganism, or allergy-related avoidance.

The term “behavior history information” refers to data indicating past actions of an individual relevant to consumption or lifestyle, including, for example, past meal-selection history, past order history from food services, frequency of high-salt food selection, and timing of meals.

The term “analysis data” refers to data obtained by preprocessing state information, preference information, and behavior history information so as to be in a format and value range that are appropriate for computational analysis and for use as input to a generative AI model.

The term “time-series statistics” refers to numerical values calculated over a temporal sequence of data points, including, for example, an average, a maximum, a minimum, a variance, a trend, or a moving average of a biometric measurement or a behavior frequency across a specified period.

The term “outlier correction” refers to processing that detects and modifies data values that deviate from expected ranges or patterns, the processing including, for example, removing or replacing abnormally large or small measurements or interpolating values based on neighboring values.

The term “normalizing numerical values” refers to transforming numeric data to a standardized scale or distribution, including, for example, scaling values to a fixed range or converting values to standardized scores, in order to stabilize subsequent processing by a model.

The term “generative AI model” refers to an artificial intelligence model configured to generate an output, such as text or structured data, based on an input including a prompt sentence, the model being implemented by machine learning techniques such as neural networks.

The term “prompt sentence” refers to a natural-language text string that specifies to a generative AI model a task, a context, and constraints for output generation, the natural-language text string being constructed from analysis data and behavior history information.

The term “proposal” refers to information generated by the generative AI model in response to a prompt sentence, the information including at least one of a meal recommendation, a drug-related suggestion, a stress-management suggestion, or an alcohol-intake restriction.

The term “preventive medicine” refers to a medical approach focusing on prevention of disease onset and progression by recommending lifestyle, dietary, or medicinal interventions based on risk factors and health status of an individual.

The term “structured data” refers to data represented in a predefined format with explicit fields, such as a record or an object, the data including at least nutrient information, intake restriction information, and emotion-correspondence information for each meal proposal item.

The term “nutrient information” refers to data indicating nutritional properties of a food item, including, for example, amounts or relative levels of energy, fat, carbohydrate, protein, sodium, vitamins, and minerals.

The term “intake restriction information” refers to data indicating recommended limits or prohibitions regarding consumption of certain components or items, such data including, for example, a low-sodium constraint, a calorie limit, or an avoidance of particular ingredients.

The term “emotion-correspondence information” refers to data indicating a relationship between a proposed food item or behavior and an emotional state, including, for example, suitability for stress reduction, mood improvement, or relaxation support.

The term “display data” refers to data generated from structured data and arranged for presentation on a user interface of a terminal, the display data including text, symbols, and layout information suitable for human perception.

The term “external food provision service” refers to a service system operated by an entity external to the claimed system, the service system being configured to receive order information for food or beverages and to manage preparation and delivery of ordered items.

The term “order information” refers to data used to request provision of at least one food or beverage item from an external food provision service, the data including at least an item identifier, a quantity, and delivery-related information.

The term “communication network” refers to an infrastructure enabling data transmission between the server of the claimed system and an external system, the infrastructure including at least one of a wired network, a wireless network, and the internet.

The term “restriction policy” refers to a set of rules or parameters specifying how intake of an alcoholic beverage should be limited or modified for an individual based on an analysis result of emotional information and optionally biometric information.

The term “alcohol-intake-restriction prompt sentence” refers to a prompt sentence that encodes a restriction policy regarding intake of an alcoholic beverage and that instructs a generative AI model to generate or adjust a proposal in accordance with such restriction policy.

The term “evaluation information” refers to information indicating an assessment of a proposal by an individual, including, for example, a rating, a preference indication, or a comment, which can be used to modify prompt configuration or model input conditions.

The term “prompt configuration” refers to a set of rules, templates, or parameters governing how a prompt sentence is composed, including selection of included fields, ordering of information, wording of instructions, and level of detail.

The term “input condition to the generative AI model” refers to a parameter or constraint with which a prompt sentence or other input is supplied to a generative AI model, including, for example, sampling temperature, maximum output length, or output format specification.

The term “stress management plan” refers to an integrated proposal that combines a meal recommendation, an exercise recommendation, and a relaxation-behavior recommendation aimed at reducing or controlling stress of an individual.

The term “high-salt-food selection tendency” refers to a tendency metric derived from behavior history information indicating a frequency or proportion of selections of food items characterized as high in salt content.

The term “late-night-eating tendency” refers to a tendency metric derived from behavior history information indicating a frequency or proportion of food consumption or orders occurring during a predetermined late-night time period.

The term “tendency information” refers to data representing at least one of a high-salt-food selection tendency and a late-night-eating tendency, the data being used as part of analysis data or for prompt generation.

In one embodiment, a server, a terminal, and a user cooperate to implement the claimed system. The server includes at least one processor, a memory storing program instructions, a network interface, and access to a persistent storage device. The terminal includes a processor, a memory, an input/output interface, and at least one sensor device such as a microphone, a camera, or a wearable-device interface. The user interacts with the terminal to provide health-related information and to receive proposals generated by the system.

Server executes a program stored in the memory. Server programmatically configures a data-acquisition module, a preprocessing module, a prompt-generation module, a generative-AI-interaction module, an output-structuring module, an external-service-integration module, an emotion-based restriction module, and a feedback-learning module. Server modules are implemented, for example, using a general-purpose programming language such as a scripting language with numerical libraries (for example, a language with array processing and data-frame libraries), and run on an operating system such as a general server operating system. Server may use a relational database system for persistent storage of user profiles and history.

Terminal executes an application that provides user interfaces for entering biometric information, emotional information, preference information, and behavior history information. Terminal may be implemented as a smartphone or tablet running a mobile operating system. Terminal application can be implemented with a native software development kit or a cross-platform framework. Terminal may communicate with wearable devices via a short-range wireless protocol to obtain heart rate, step count, and other sensor measurements.

User operates the terminal application to enter values such as age, sex, weight, height, and textual descriptions of current mood. User may also respond to questionnaires regarding dietary preferences and typical behavior, such as frequency of late-night meals or consumption of high-salt food. User may allow the terminal to capture audio and video samples, which include voice tone and facial expression, so that emotional information can be inferred.

Server receives biometric information and emotional information from the terminal through the network interface. Server stores the received information in structured records, for example in tables or documents that include fields for raw measurements, timestamps, and source devices. Server uses a data model in which a “health profile” record references biometric attributes and derived indicators, and an “emotion profile” record references emotional categories and confidence scores.

Server preprocesses state information, preference information, and behavior history information to create analysis data suitable for use with a generative AI model. Server uses a data-frame library to convert raw records into tabular form, where rows represent time-stamped events and columns represent quantitative features such as systolic blood pressure, diastolic blood pressure, heart rate, step count, estimated calorie intake, and categorical codes for meal type. Server computes time-series statistics such as moving averages, trends, maxima, and minima by applying rolling-window and aggregation operations. Server performs outlier correction by applying statistical thresholds or robust measures (for example, interquartile ranges) and replacing anomalous values with interpolated or clipped values. Server performs normalization by scaling numeric columns to standardized ranges or distributions, which reduces dynamic range and improves numerical stability in subsequent model processing.

Server differentiates between preference information and behavior history information.

Server encodes preference information, such as favorite cuisines or disliked ingredients, using categorical encoding or binary flags. Server encodes behavior history information, such as occurrence of high-salt meals or time of day of orders, into numeric counts, frequencies, and temporal pattern features. Server in one embodiment constructs specific tendency measures, such as a high-salt-food selection tendency and a late-night-eating tendency, by dividing counts of high-risk events by total events over a defined period. This creates low-dimensional features that summarize complex behavioral patterns and allow the system to compute these tendencies efficiently at query time.

Server constructs a feature vector that includes biometric statistics, emotional indicators, preference encodings, and behavior-tendency metrics. Server stores this feature vector as analysis data in memory. Server uses this analysis data both for constructing prompt sentences and, in certain embodiments, for direct numeric input into a neural network that forms part of the generative AI model.

Server analyzes emotional information by applying specific algorithms. Server may use a speech-to-text engine to convert captured audio into a text transcript, then uses a natural-language processing model to compute sentiment scores and categorize emotional state. Server may use a vision-processing library to analyze facial images and determine facial action units or expression labels, which are then mapped to discrete emotional classes such as “high stress” or “relaxed.” Server may use an internal rule set or a trained classifier that combines textual sentiment, audio prosody measures (pitch, loudness, speaking rate), and facial expression labels into a unified emotion score. The use of multiple modalities and an internal fusion algorithm allows server to create more robust emotion estimates than manual inspection, and server can tune thresholds and weights for each modality to optimize classification accuracy.

Server generates a prompt sentence for interaction with a generative AI model. Server uses a prompt-generation module that programmatically composes natural-language text from analysis data. Server defines prompt templates that include placeholders for health profile summaries, preference information, behavior tendencies, and emotion states. Server fills the placeholders with specific values from the current analysis data, and orders segments of the sentence according to a configuration that has been optimized based on prior feedback.

Unlike static hand-written prompts, this dynamic composition allows the server to tailor the content and structure of the prompt to the current individual state and to the generative AI model's expected input format, thereby reducing ambiguity and improving the model's ability to produce targeted output.

Server may generate, for example, a prompt sentence such as:
“User: male, 50 years old, BMI 28, diagnosed hypertension, family history of diabetes.
Latest readings: average systolic blood pressure 145 mmHg, average daily steps 4,000, average sleep 6 hours per night.
Preferences: likes Japanese food, dislikes fried food, no known allergies.
Behavior: frequently orders high-salt meals late at night.
Emotion state: high stress detected from voice and text.
Goal: preventive medicine.
Task: As a medical-nutrition assistant, generate a low-sodium, heart-healthy dinner plan for tonight and suggestions for stress-reduction activities. Avoid recommending alcohol.” Server may also generate a specialized alcohol-intake prompt sentence such as:
“Given that the user has hypertension and is currently under high stress, generate a recommendation that limits or avoids alcohol consumption tonight and proposes non-alcoholic alternatives.”

Server may further generate a prompt sentence for a stress-management plan, for example:
“Based on the user's health information and emotional state, generate a one-week stress management plan including meal recommendations, exercise recommendations, and relaxation activities suitable for a high-stress individual with elevated blood pressure.”

Server sends the prompt sentence to a generative AI model. In one embodiment, the generative AI model is implemented as a neural-network-based language model having multiple layers of attention blocks and feedforward units. Server may call such a model hosted on a separate computation environment through a network interface, or may execute a locally hosted model implemented using a machine-learning framework. The model may be trained on a large corpus of text containing health and nutrition content, and fine-tuned on domain-specific data, such as clinical guidelines or diet recommendations, with special tokens for representing constraints like “low sodium” or “high fiber.” The model internally uses tokenization to convert the prompt sentence into token indices, performs parallel matrix multiplications across its layers, and generates a sequence of output tokens by sampling from probability distributions determined by its output layer.

Server controls model parameters such as sampling temperature, maximum output length, and output format hints at each invocation. Server uses an input condition configuration that specifies, for example, that outputs should be concise, structured into sections (meal plan, rationale, lifestyle advice), or returned in a format that can be parsed more easily. Server sets these parameters based on prompt configuration and resource constraints, which allows the system to balance generation quality and computational cost.

Server receives the output from the generative AI model and processes it using the output-structuring module. Server parses the textual output, using pattern recognition and, in some embodiments, a secondary parsing model, to identify specific meal proposal items, descriptions, and any nutritional commentary included by the model. Server maps meal proposal items into structured records, each with fields for name, ingredients, nutritional attributes, and tags such as “low sodium” or “suitable for high stress.” Server adds emotion-correspondence information based on either explicit statements in the output or derived rules, for example if a plan mentions herbal teas or foods high in certain vitamins known to support mood.

Server associates structured meal proposal items with item information from an external food provision service. Server maintains a catalog that includes external item identifiers, names, and nutritional metadata. Server uses string matching, semantic similarity scoring, or explicit mapping tables to match proposed items to catalog entries. Server generates order information that identifies the external items, quantities, and user delivery details, and sends this information through a communication network using a service-specific interface. This integration allows the system to bridge the gap between AI-generated proposals and actual provisioning of food items.

Server determines a restriction policy for alcohol intake by applying rules to emotion and health features. Server may define a restriction policy as a small structure containing fields such as “allowed”, “maximum units”, and “permitted time window.” Server sets these fields based on thresholds and conditions, for example if emotion state indicates high stress and health profile indicates hypertension, server may set allowed to false. Server encodes this restriction policy in an alcohol-intake-restriction prompt sentence and sends it to the generative AI model, which results in the model generating proposals that exclude alcoholic beverages and instead include alternatives. The explicit use of a structured restriction policy and a dedicated prompt ensures that proposals adhere consistently to the policy and reduces the risk of inconsistent textual guidance.

Server acquires evaluation information from the user after proposals have been displayed and optionally executed. User may rate proposals using predefined scales or add free-text comments. Terminal sends this evaluation information to the server, which stores it and associates it with the corresponding analysis data and prompt configuration. Server uses data-frame analysis and aggregation to compute statistics such as average rating per prompt template, per emotion state, or per health condition. Server may train a separate model or apply heuristic optimization to determine which prompt structures and parameter settings produce higher satisfaction or better adherence to constraints. Server updates prompt templates and input conditions accordingly. For example, if dropping certain details in the prompt yields ambiguous outputs and lower ratings, server reinserts such details into future prompts. This feedback-driven adaptation of prompts and conditions improves the effectiveness and efficiency of subsequent generative AI interactions.

Terminal displays proposals and status information to the user. Terminal converts the structured data and display data received from the server into graphical elements such as text blocks, icons, and lists of menu options. Terminal highlights key attributes, such as low-sodium labels or stress-relief indicators, to guide user attention. Terminal further displays external service order status, using identifiers and timestamps received from the server. Terminal thus acts as the interface between the server's internal computation and the user's real-world actions.

This architecture provides technical effects beyond mere automation of human mental tasks or business processes. Server uses a multi-stage data pipeline that transforms heterogeneous, noisy, and partially missing real-world signals into compact numeric feature vectors optimized for generative AI processing. This preprocessing reduces the dimensionality and variability of the input space, which improves computational stability and reduces the number of tokens needed in the prompt sentences. By generating context-adaptive prompt sentences and controlling model parameters, server avoids repeatedly sending redundant or extraneous information, which reduces communication load and computation time at the generative AI model. The mapping of generative AI outputs into structured data and their reuse in downstream modules, such as external service integration and feedback analysis, allow the system to function as a coordinated computer network rather than isolated human-in-the-loop steps.

Server performs specific non-conventional operations that are not simple computerization of mental reasoning. For example, server computes behavior tendencies by analyzing time-stamped order logs and extracting frequency metrics over sliding time windows using optimized vector operations. This type of data handling is designed for large-scale computational efficiency and is not readily performed manually. The prompt-generation module optimizes the order and selection of textual fields based on feedback-derived statistics, which is a machine-internal adaptation mechanism that reduces model error rates and improves convergence of generated output quality. The feedback-learning module closes the loop by updating configuration parameters and templates automatically, yielding a progressive improvement in recommendation precision and stability.

In additional embodiments, server can use different types of neural network architectures for the generative AI model, such as encoder-decoder models, and can vary the training method, for example by using supervised fine-tuning on paired prompt-response data, reinforcement learning from human feedback, or multi-task learning that jointly optimizes for diet recommendation and stress-management tasks. Server can vary the error function for training, for example combining language modeling loss with penalty terms for violating constraints like sodium limits. Server can incorporate data augmentation techniques such as paraphrasing prompts and adding noise to numeric values within safe bounds, which make the model more robust to variations in input phrasing and measurement errors.

Alternative embodiments may adjust modules while maintaining the core concept. For example, server may implement emotion analysis entirely on the terminal using specialized hardware accelerators and send only emotion state labels to the server. Server may implement the external food provision service interface using message queues instead of direct HTTP calls. Server may employ different database technologies, such as document-oriented storage, if the structure of the health profile and behavior history evolves. In all such variations, server continues to acquire and preprocess state information, construct and refine prompt sentences, interact with a generative AI model, convert outputs into structured data, integrate with external services, and adapt prompt configuration based on user evaluation.

Through these specific technical structures and operations, the system improves functioning of the computer as a whole. Data preprocessing and feature extraction reduce computational burden and latency. Dynamic prompt generation and feedback-based configuration improve accuracy and consistency of generative AI responses. Structured mapping of AI output into machine-processable formats enables reliable automation of downstream technical processes, including external device and service control. Emotion-based restriction policies encoded in prompts yield controlled AI behavior that adheres to health and safety constraints. As a result, the system achieves improved processing speed, enhanced precision of generated proposals, reduced error in mapping from AI text to real-world actions, and more efficient use of network and computational resources compared to conventional static or rule-based systems.

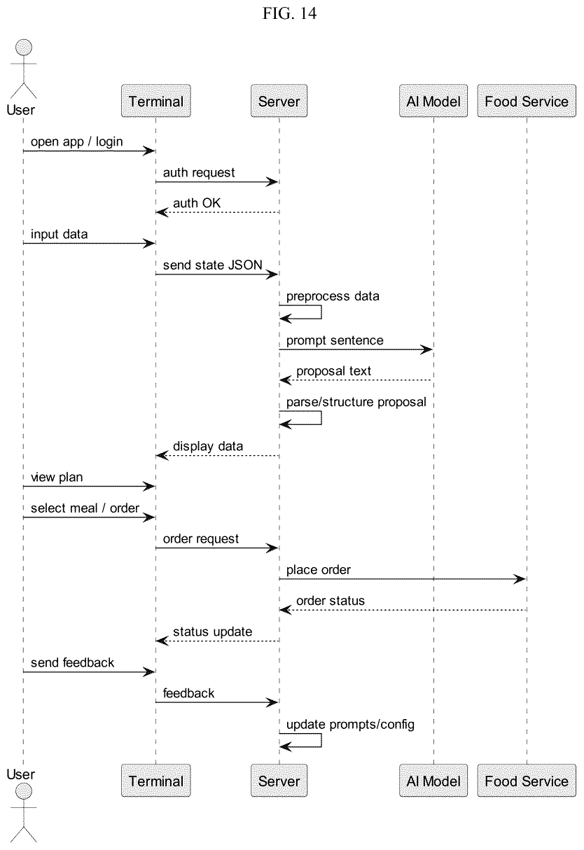

The following describes the processing flow using FIG. 14.

### Step 1:

User launches the application on the terminal and logs in.

Terminal displays input screens for biometric information, emotional information, preference information, and behavior history information.

Input: existing user identifier or new registration information entered by the user.

Output: authenticated session token and initial empty forms for data input.

Terminal sends the authentication request and receives the session token from the server.

### Step 2:

User enters biometric information such as age, sex, weight, height, blood pressure values, and known medical conditions into the terminal.

Input: user-typed values in text fields, selection boxes, and checkboxes.

Output: a local data structure on the terminal that holds raw biometric information.

Terminal validates formats (for example, numeric ranges) and stores the values in a temporary object.

### Step 3:

User inputs emotional information and preferences through the terminal.

Input: user's mood selection (for example, stress level sliders), text describing current feelings, answers to preference questions, and optionally consent to audio/video capture.

Output: a local data structure containing emotional information and preference information. Terminal may activate the microphone and camera, capture short audio/video samples, and attach metadata such as capture time and device ID.

### Step 4:

Terminal aggregates biometric information, emotional information, preference information, and behavior history information.

Input: temporary objects holding biometric data, emotional data, preference data, and any local logs of past selections or meals.

Output: a unified JSON object containing fields such as “health_info”, “emotion_info”, “preferences”, and “behavior_history”.

Terminal packs the data into JSON, adds the session token, and prepares an HTTPS request.

### Step 5:

Terminal transmits the JSON object to the server.

Input: unified JSON object and server endpoint address.

Output: network request sent to the server over a secure communication channel.

Terminal uses HTTPS, sets appropriate headers, and sends the payload to an API endpoint exposed by the server.

### Step 6:

Server receives and parses the request.

Input: HTTPS request containing the JSON payload and authentication token.

Output: parsed data structures representing raw state information and a validation result.

Server authenticates the token, decodes the JSON using a parsing library, and checks that required fields are present and within plausible ranges.

### Step 7:

Server stores raw state information in persistent storage.

Input: validated biometric information, emotional information, preference information, and behavior history information.

Output: database records in tables or collections such as “health_profile”, “emotion_profile”, “preferences”, and “history”.

Server issues database insert or update commands, associating all records with the user identifier and timestamps.

### Step 8:

Server retrieves recent history and constructs a time-series dataset.

Input: user identifier and time range (for example, last 30 days), used as query parameters.

Output: a time-ordered list of measurements and events containing values such as blood pressure, heart rate, step counts, and meal logs.

Server executes database queries and loads the results into a data-frame structure in memory.

### Step 9:

Server preprocesses numeric biometric and behavior data.

Input: data-frame rows containing raw numeric measurements and event times.

Output: a cleaned and normalized data-frame suitable as analysis data.

Server removes or replaces outliers using statistical rules, interpolates missing values, and applies normalization (for example, scaling to zero mean and unit variance).

### Step 10:

Server computes time-series statistics and derived indicators.

Input: cleaned data-frame of measurements and events.

Output: aggregated features such as average blood pressure, trend of weight, mean step count, and moving-average heart rate.

Server applies rolling windows and aggregation functions to derive per-user statistics, and calculates body mass index and other derived measures.

### Step 11:

Server encodes preference information and behavior tendencies.

Input: stored preference responses, food-selection logs, and order history.

Output: encoded preference vector and tendency metrics such as high-salt-food selection tendency and late-night-eating tendency.

Server counts specific event types over defined periods, divides counts by total events to obtain frequencies, and maps categorical preferences to binary or multi-class encodings.

### Step 12:

Server analyzes emotional information.

Input: emotional data from text, audio, and video sources, including any transcripts and expression labels.

Output: a unified emotion state label and associated confidence scores.

Server applies sentiment-analysis algorithms to text, extracts prosodic features from audio, classifies facial expressions from images, and combines these signals using weighted rules or a classifier to determine states such as “high stress” or “relaxed”.

### Step 13:

Server constructs an analysis feature set.

Input: aggregated biometric features, encoded preferences, behavior tendencies, and emotion state label.

Output: an analysis feature vector or structured object used for prompt generation.

Server concatenates numeric arrays and categorical codes into a single structure and stores it in memory for the current session.

### Step 14:

Server determines an alcohol-intake restriction policy.

Input: analysis features including blood pressure, medical conditions, and emotion state.

Output: a restriction policy object specifying whether alcohol is allowed and any quantitative limits.

Server applies predefined rules that evaluate thresholds, for example disallowing alcohol when high blood pressure and high stress coexist, and populates policy fields accordingly.

### Step 15:

Server generates a main prompt sentence for the generative AI model.

Input: analysis feature set, user goal (for example, preventive medicine), and configuration information for prompt templates.

Output: a natural-language prompt sentence describing the user condition and requested task.

Server fills a template with specific values, orders segments according to a configuration, and concatenates text portions to produce a coherent prompt.

### Step 16:

Server generates a specialized alcohol-restriction prompt sentence.

Input: restriction policy object and emotion state.

Output: an alcohol-intake-restriction prompt sentence instructing the generative AI model to limit or avoid alcohol.

Server encodes policy parameters into descriptive text, such as “avoid recommending alcohol tonight” or “limit alcohol to one small unit”, and appends context about stress level and health status.

### Step 17:

Server sends the prompt sentence or sentences to the generative AI model.

Input: constructed prompt sentences and model input parameters such as maximum tokens and sampling temperature.

Output: model request objects containing tokenized prompts sent to the generative AI model.

Server tokenizes the text (if local model is used) or packages the text and parameters in an API request (if remote model is used) and transmits the request.

### Step 18:

Server receives generated proposals from the generative AI model.

Input: model responses representing sequences of tokens or text strings.

Output: plain-text proposals that include meal recommendations, possible drug-related suggestions, and lifestyle or stress-management advice.

Server decodes tokens into text if necessary, concatenates segments, and performs basic validity checks such as length and presence of required sections.

### Step 19:

Server parses and structures the generated proposals.

Input: plain-text proposals containing narrative descriptions and lists of foods or actions.

Output: structured data records for each meal proposal item and associated advice.

Server uses pattern matching, delimiters, or a secondary parsing model to identify dish names, ingredients, nutritional claims, and instructions, and populates structured objects with this information.

### Step 20:

Server enriches structured data with nutrient, restriction, and emotion-correspondence information.

Input: structured meal items and a nutritional database or ruleset.

Output: augmented meal items with explicit nutrient fields, intake restriction flags, and emotion-support tags.

Server queries a nutritional data source for nutrient values, sets flags for low sodium or high vitamin content, and labels items as suitable for stress reduction when matching certain criteria.

### Step 21:

Server maps structured meal items to external food provision service items.

Input: enriched structured meal items and catalog entries from external services.

Output: mappings between internal meal identifiers and external item identifiers.

Server performs matching based on names and attributes, resolves ambiguities using similarity scores, and records the mapping in a table.

### Step 22:

Server generates order information for external services.

Input: mapped meal items, user delivery information, and selected quantities.

Output: order information objects containing external item IDs, quantities, and delivery parameters.

Server composes order payloads conforming to the external service's API specification and prepares them for transmission.

### Step 23:

Server constructs display data for the terminal.

Input: enriched structured proposals, alcohol-restriction guidance, and optional exercise or relaxation recommendations.

Output: display data describing sections, titles, text content, icons, and emphasis tags.

Server arranges proposals into logical groups, marks key attributes, and formats text into short paragraphs and bullet-like structures ready for rendering.

### Step 24:

Server sends the display data and any initial order options to the terminal.

Input: display data and mappings for orderable items.

Output: an HTTPS response containing JSON-formatted display and ordering information.

Server serializes the data and includes necessary identifiers so the terminal can later reference specific items and proposals.

### Step 25:

Terminal receives and renders the display data.

Input: JSON response from the server containing proposals, tags, and order options.

Output: graphical user interface screens showing recommended meals, restrictions, and lifestyle suggestions.

Terminal parses the JSON, creates UI components for lists and text, and visually emphasizes low-sodium tags and alcohol warnings.

### Step 26:

User reviews proposals and optionally selects meals to order.

Input: rendered proposals and interactive elements such as buttons and checkboxes.

Output: user selections of specific meals, quantities, and desired delivery time windows.

User taps on items or adjusts settings on the terminal application.

### Step 27:

Terminal sends the user's order selections to the server.

Input: identifiers of selected internal meal items, requested quantities, and delivery preferences.

Output: an order-request JSON object transmitted to the server.

Terminal composes a request including the session token and sends it over HTTPS.

### Step 28:

Server processes the order request and contacts external food provision services.

Input: order-request JSON object, internal-to-external item mapping, and user delivery information from storage.

Output: outgoing order messages addressed to external services and a local record of the order.

Server fills external API fields, transmits the payloads, and records order IDs and statuses received in responses.

### Step 29:

Server returns order confirmation and tracking information to the terminal.

Input: order status responses from external services including confirmation codes and estimated delivery times.

Output: confirmation and tracking data in a structured response to the terminal.

Server associates external status with internal order records and packages it into a response.

### Step 30:

Terminal displays order status and updates it as needed.

Input: confirmation and tracking data from the server.

Output: on-screen indicators such as “Order confirmed”, “Preparing”, and “Out for delivery” with time estimates.

Terminal periodically requests updated status or listens for push messages and refreshes the display.

### Step 31:

User consumes the proposals as guidance, with or without ordering.

Input: recommended meals, restrictions, and lifestyle suggestions shown on the terminal.

Output: user decisions to follow suggested meals, adjust diet, or perform recommended exercises or relaxation activities.

User may use the information for home cooking or personal planning.

### Step 32:

User provides evaluation information through the terminal.

Input: ratings, comments, or feedback forms concerning the usefulness and suitability of the proposals.

Output: feedback data captured by the terminal for transmission.

Terminal records numeric ratings and free-text entries and ties them to specific proposal identifiers.

### Step 33:

Terminal sends the evaluation information to the server.

Input: feedback data and identifiers of the related prompt configuration or session.

Output: a feedback JSON object sent to the server via HTTPS.

Terminal ensures that user and session identifiers are included for correct association.

### Step 34:

Server stores and analyzes feedback data.

Input: feedback JSON object containing ratings and comments, plus metadata about prompt templates and model parameters used.

Output: updated feedback records in the database and aggregated statistics over time.

Server inserts feedback into a dedicated table, periodically runs aggregation queries, and computes metrics such as average rating per prompt template or per health condition.

### Step 35:

Server updates prompt configuration and model input conditions based on feedback.

Input: feedback statistics, error patterns, and correlation between prompt structures and user satisfaction.

Output: revised prompt templates, parameter settings (such as model temperature and maximum length), and configuration rules used in future sessions.

Server adjusts which fields are emphasized, how instructions are phrased, and how strict constraint descriptions are, thereby iteratively improving prompt sentences and generative AI model interaction for subsequent executions.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### SECOND EXEMPLARY EMBODIMENT

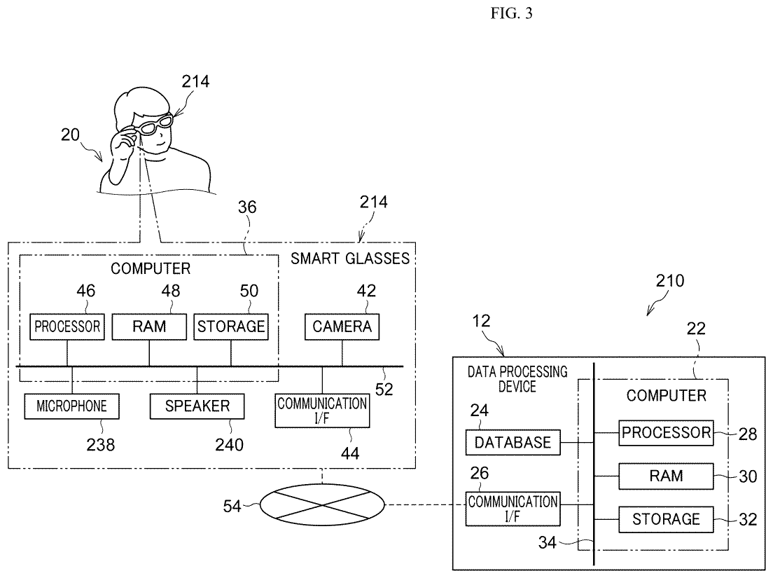

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

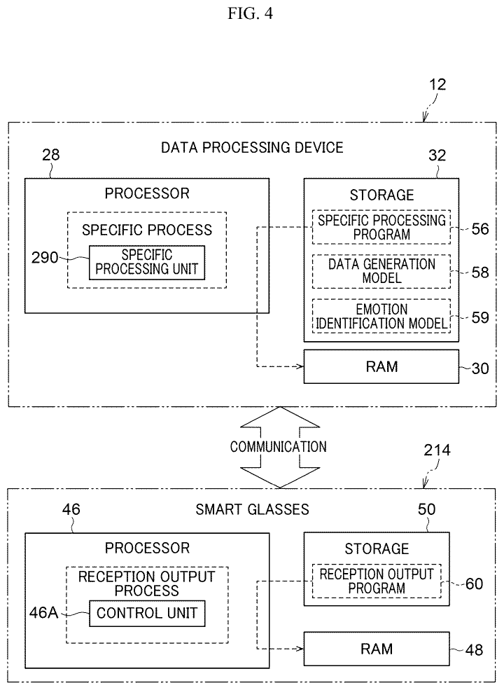

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### THIRD EXEMPLARY EMBODIMENT

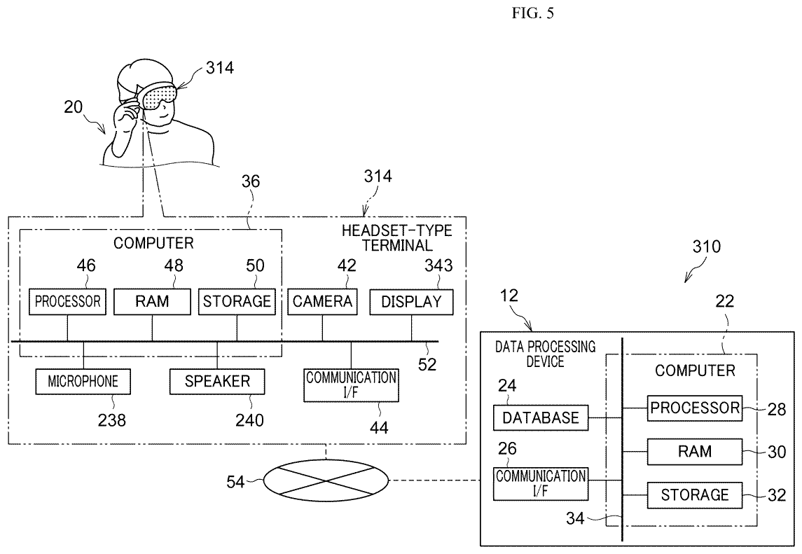

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

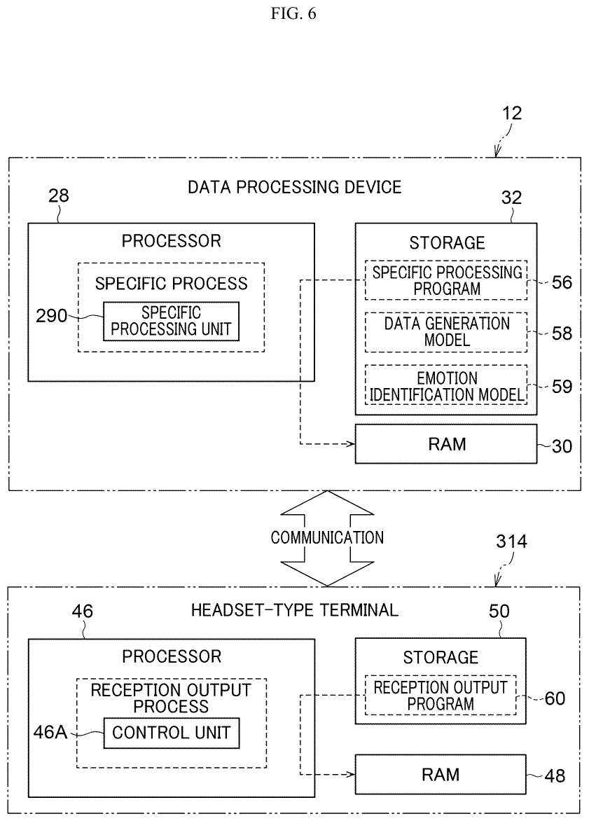

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### FOURTH EXEMPLARY EMBODIMENT

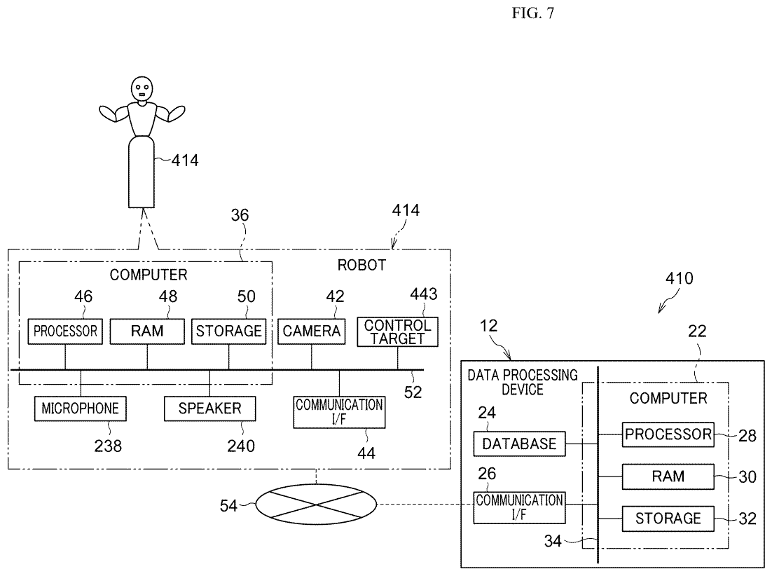

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

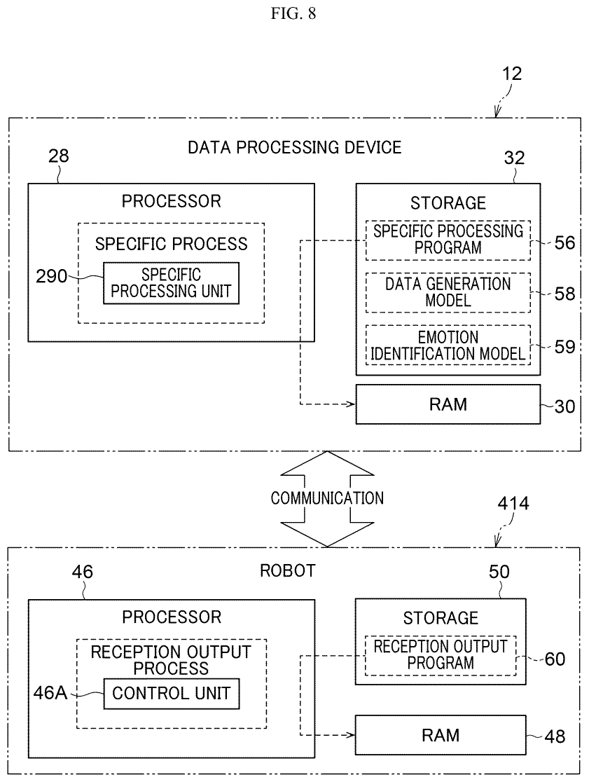

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

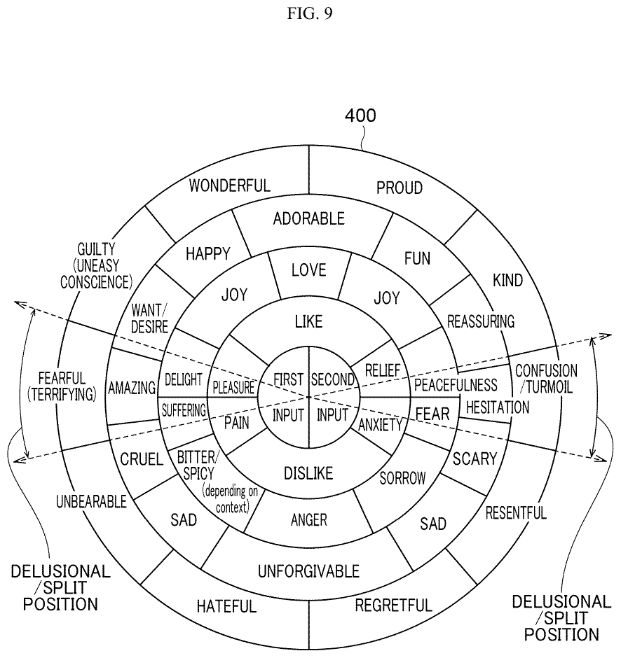

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University).

Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

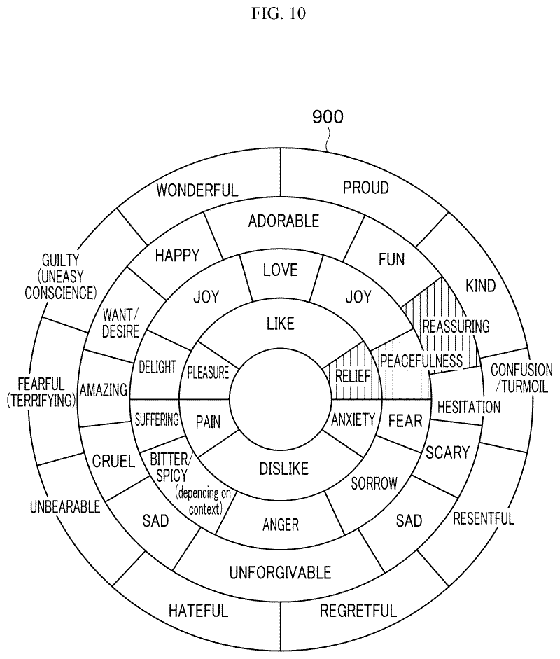

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire individual health-related information and emotional-related information via a user interface of an information processing terminal and transmit the individual health-related information and the emotional-related information to the system through a communication network,
store the transmitted health-related information and emotional-related information as structured information in a storage device of an information processing apparatus, perform statistical computation processing on the stored health-related information, and generate an analysis result including a body index, a risk index, and a lifestyle index,
generate, on the basis of the analysis result, a prompt sentence for a generative AI model, the prompt sentence describing in natural language instructions related to nutrient intake, ingestion of substances, execution of physical activity, improvement of lifestyle habits, and adjustment of emotional states from the viewpoint of preventive healthcare,
transmit the prompt sentence for the generative AI model as a response generation request to an external generative AI model and acquire a natural language response from the external generative AI model,
extract, from the acquired natural language response, a meal proposal, an ingestion substance proposal, a physical activity proposal, a stress management proposal, and an alcohol intake restriction proposal according to the emotional-related information, and convert the extracted proposals into display information for presentation to an individual, generate cooperation information that enables selection and ordering of proposed foodstuffs by performing communication with an external goods delivery service on the basis of meal proposal information included in the display information, and
additionally generate, on the basis of the emotional-related information and the result of the statistical computation processing, a further prompt sentence for a generative AI model regarding relaxation or strengthening of alcohol intake restrictions, and dynamically update the alcohol intake restrictions by using a response from the generative AI model.

### (Supplementary 2)

The system according to supplementary 1,
wherein the processor is configured to generate, on the basis of the analysis result and the emotional-related information, a prompt sentence for a generative AI model including proposals relating to stress reduction actions,
sleep habit adjustment, and psychological self-care, and provide to the individual a response relating to stress management output from the generative AI model.

### (Supplementary 3)

The system according to supplementary 1,
wherein the processor is configured to
generate, on the basis of the body index and the risk index obtained by the statistical computation processing and on the basis of the emotional-related information, a prompt sentence for a generative AI model that enumerates in natural language a group of food items recommended for intake and a group of food items to be restricted, and automatically generate order candidates for the group of food items by cooperation with the external goods delivery service using food candidate information obtained from the generative AI model.

### Application Example 1

### (Supplementary 1)

A system comprising a processor,
wherein the processor is configured to
receive, from a communication terminal, input information including medical information of an individual and an emotional state of the individual,
preprocess the input information including the medical information and the emotional state,
generate a prompt sentence describing the input information, and input the prompt sentence or preprocessed feature data into a generative artificial intelligence model to cause the generative artificial intelligence model to generate a health evaluation result that classifies a health condition of the individual into a plurality of stages and to generate benefit information based on the health evaluation result,
generate payment benefit data including at least one of a discount rate and an economic privilege to be applied in an electronic payment process, based on the health evaluation result and the benefit information obtained from the generative artificial intelligence model, and transmit the payment benefit data to an external payment processing device or to the communication terminal,
transmit display data including the health evaluation result and the benefit information to the communication terminal, and cause the communication terminal to present, to the individual, proposals and benefits corresponding to the health evaluation result and the benefit information, and
generate a prompt sentence including instruction content relating to at least one of dietary proposals, medication proposals, stress management proposals, and adjustment of alcohol intake restriction, the instruction content being based on the medical information of the individual and the emotional state of the individual, input the prompt sentence into the generative artificial intelligence model to cause the generative artificial intelligence model to generate the proposals, and enable cooperation with an external service providing device to execute at least one of order processing and restriction adjustment processing based on the proposals.

### (Supplementary 2)

The system according to supplementary 1,
wherein the processor is configured to calculate, based on the health evaluation result and the benefit information, a discounted amount by applying the discount rate to a payment amount in the electronic payment process, and to output the discounted amount and the applied benefit information to at least one of the communication terminal and the external payment processing device.

### (Supplementary 3)

The system according to supplementary 1,
wherein the processor is configured to generate the prompt sentence to be input into the generative artificial intelligence model as a natural language sentence including a description part that enumerates, item by item, the medical information of the individual and the emotional state of the individual, and an instruction part that specifies output formats relating to at least the health evaluation result, the benefit information, the dietary proposals, the medication proposals, the stress management proposals, and the adjustment of the alcohol intake restriction.

### Example 2

### (Supplementary 1)

A system comprising a processor,
wherein the processor is configured to
receive, from a terminal device, personal biological information and emotional information of an individual as structured data,
perform numerical processing and statistical processing on the structured data to calculate health indices and risk indices and to extract features based on the health indices and the risk indices,
generate a prompt sentence, based on the biological information, the emotional information, and the extracted features, for causing a generative AI model to generate proposals relating to at least one of nutritional intake, exercise, and medicinal use in preventive medicine, and input the prompt sentence into an external generative information processing model to obtain proposal information,
transmit the proposal information as visual information to the terminal device to present the proposal information to the individual, and cooperate with an external goods supply service to enable ordering of food, beverages, or lifestyle-related goods based on the proposal information, and
generate a prompt sentence, based on the emotional information and the extracted features, for adjusting at least one of an alcohol intake limit amount and an alcohol intake frequency, input the prompt sentence into the generative information processing model, and update the proposal information based on an adjustment result obtained from the generative information processing model.

### (Supplementary 2)

The system according to supplementary 1,
wherein the processor is configured to
generate, based on the extracted features, a prompt sentence for the generative information processing model to generate stress management proposals relating to at least one of stress-relief behaviors, sleep habits, and lifestyle habit improvement, and to include the stress management proposals in the proposal information.

### (Supplementary 3)

The system according to supplementary 1,
wherein the processor is configured to
generate, based on the extracted features and the biological information, a prompt sentence for the generative information processing model to generate proposal information that specifically enumerates at least one of a group of foods and a group of beverages suitable for the individual, and to associate items included in the group of foods and the group of beverages with items provided by the external goods supply service so as to present order candidates.

### Application Example 2

### (Supplementary 1)

A system comprising a processor,
wherein the processor is configured to
acquire biometric information and emotional information of an individual and store the biometric information and the emotional information as state information,
preprocess the state information, preference information, and behavior history information of the individual as analysis data, the preprocessing including calculating time-series statistics, correcting outliers, and normalizing numerical values,
generate, based on the analysis data and the behavior history information, a prompt sentence in a natural language for causing a generative AI model to generate a proposal related to a meal or a drug based on preventive medicine, and cause the generative AI model to input the prompt sentence,
analyze an output proposal from the generative AI model, convert a meal proposal item in the output proposal into structured data including nutrient information, intake restriction information, and emotion-correspondence information, and convert the structured data into display data to be presented to the individual,
associate the meal proposal item in the structured data with item information of an external food provision service, generate order information for food based on the proposal, and transmit the order information to the external food provision service via a communication network to enable ordering by the individual,
determine a restriction policy regarding intake of an alcoholic beverage based on an analysis result of the emotional information, generate an alcohol-intake-restriction prompt sentence reflecting the restriction policy, and cause the generative AI model to input the alcohol-intake-restriction prompt sentence to adjust the proposal, and
acquire evaluation information from the individual regarding the proposal and update at least one of a configuration of the prompt sentence and an input condition to the generative AI model based on the evaluation information.

### (Supplementary 2)

The system according to supplementary 1,
wherein the processor is configured to
generate, by the prompt sentence, an instruction to the generative AI model to generate a stress management plan including a meal proposal, an exercise proposal, and a relaxation-behavior proposal based on the biometric information, the emotional information, and the preference information of the individual.

### (Supplementary 3)

The system according to supplementary 1,
wherein the processor is configured to calculate, in the preprocessing of the analysis data, at least one of a high-salt-food selection tendency and a late-night-eating tendency from past meal-selection history or order history as the behavior history information, provide tendency information to a prompt generation process, and cause the generative AI model to generate a prompt sentence that instructs prioritization of proposals for low-salt food and food based on preventive medicine.

What is claimed is:

1. A system comprising:
circuitry configured to:
receive, via a communication interface coupled to a packet-switched network, first attribute data comprising health-related parameters and second attribute data comprising emotional-state parameters from a terminal device, and store the first attribute data and the second attribute data as structured data in a storage device;
apply a statistical computation algorithm to the stored first attribute data to generate an analysis result comprising a body index, a risk index, and a lifestyle index;
automatically generate, based on the analysis result, a prompt sentence for a generative neural network model, the prompt sentence specifying in natural language instructions related to at least nutrient intake parameters, substance ingestion parameters, physical activity parameters, lifestyle adjustment parameters, and constraint parameters conditioned on the second attribute data;
transmit the prompt sentence to the generative neural network model via the communication interface, receive a natural language response from the generative neural network model, and parse the natural language response to extract at least a first proposal dataset, a second proposal dataset, a third proposal dataset, a fourth proposal dataset, and a constraint proposal dataset;
convert the extracted proposal datasets into display data and transmit the display data to the terminal device; and
generate a further prompt sentence for the generative neural network model based on the second attribute data and the analysis result, transmit the further prompt sentence to the generative neural network model, and dynamically update constraint control data based on a response from the generative neural network model.

2. The system according to claim 1, wherein the circuitry is configured to compute the body index from biometric parameters included in the first attribute data, compute the risk index from a combination of the body index and lifestyle parameters, and compute the lifestyle index from activity frequency parameters and behavioral pattern parameters derived from the stored first attribute data.

3. The system according to claim 2, wherein the circuitry is configured to generate the prompt sentence by selecting a prompt template based on a proposal type, substituting the body index, the risk index, the lifestyle index, and the second attribute data into the prompt template, and appending output format specifications defining a response structure comprising labeled proposal sections.

4. The system according to claim 3, wherein the circuitry is configured to parse the labeled proposal sections of the natural language response using a structural extraction algorithm that identifies section boundaries, extract the first proposal dataset, the second proposal dataset, the third proposal dataset, and the fourth proposal dataset from respective labeled sections, and store the extracted datasets as structured records in the storage device.

5. The system according to claim 4, wherein the circuitry is configured to generate stress management proposal data from the fourth proposal dataset, incorporate the stress management proposal data into the display data, and transmit the display data to the terminal device for presentation.

6. The system according to claim 1, wherein the circuitry is configured to receive updated first attribute data from the terminal device at a predetermined update interval, recompute the body index, the risk index, and the lifestyle index from the updated first attribute data, regenerate the prompt sentence incorporating the updated analysis result, and supply the regenerated prompt sentence to the generative neural network model.

7. The system according to claim 6, wherein the circuitry is configured to compare the updated analysis result against stored analysis results from prior processing cycles, compute a trend metric indicating a direction and magnitude of change, and incorporate the trend metric into the prompt sentence as an additional analysis condition.

8. The system according to claim 1, wherein the circuitry is configured to detect a change in the second attribute data exceeding a state-change threshold between consecutive processing cycles, regenerate the further prompt sentence incorporating the updated second attribute data, transmit the regenerated further prompt sentence to the generative neural network model, and update the constraint control data based on the regenerated response.

9. The system according to claim 8, wherein the circuitry is configured to classify the second attribute data into one of a plurality of emotional state categories, select a constraint adjustment strategy from a plurality of constraint adjustment strategies based on the classified emotional state category, and incorporate the selected constraint adjustment strategy as a constraint specification in the further prompt sentence.

10. The system according to claim 1, wherein the circuitry is configured to generate cooperation data based on the first proposal dataset, transmit the cooperation data to an external goods delivery interface via the communication interface to enable selection of proposed item data, receive order confirmation data from the external goods delivery interface, and store the order confirmation data in the storage device.

11. The system according to claim 10, wherein the circuitry is configured to generate the cooperation data by extracting item identifiers and quantity parameters from the first proposal dataset, formatting the item identifiers and quantity parameters into a structured request data format conforming to an interface specification of the external goods delivery interface, and transmitting the structured request data via the communication interface.

12. The system according to claim 1, wherein the circuitry is configured to store a history of analysis results, generated prompt sentences, natural language responses, and constraint control data in the storage device indexed by a user identifier and a timestamp, and supply stored history data as context in subsequent prompt sentence generation to improve longitudinal consistency of proposals.

13. The system according to claim 12, wherein the circuitry is configured to apply a trend analysis algorithm to the stored history data to detect statistically significant changes in the body index or risk index over time, incorporate the detected trends as longitudinal context in subsequently generated prompt sentences, and generate updated proposal datasets reflecting the longitudinal trends.

14. The system according to claim 1, wherein the circuitry is configured to receive a natural language inquiry from the terminal device, generate an inquiry-response prompt sentence incorporating the inquiry and the analysis result, transmit the inquiry-response prompt sentence to the generative neural network model, and receive an inquiry-specific response for transmission to the terminal device.

15. The system according to claim 14, wherein the circuitry is configured to apply a hallucination detection algorithm to the inquiry-specific response by comparing key parameter terms in the response against a reference parameter lexicon stored in the storage device, flag response segments containing unrecognized parameter terms, and regenerate flagged response segments by supplying a correction prompt sentence to the generative neural network model.

16. The system according to claim 1, wherein the circuitry is configured to receive physical activity sensor data from the terminal device, incorporate the physical activity sensor data into the first attribute data as an activity parameter, recompute the lifestyle index based on the updated first attribute data, and regenerate the prompt sentence incorporating the updated lifestyle index.

17. The system according to claim 1, wherein the circuitry is configured to generate a risk-alert prompt sentence incorporating the risk index and a risk threshold stored in the storage device, transmit the risk-alert prompt sentence to the generative neural network model when the risk index exceeds the risk threshold, and receive a risk-alert response for transmission to the terminal device with higher transmission priority than the display data.

18. A system comprising:
circuitry configured to:
receive, via a communication interface coupled to a packet-switched network, first attribute data comprising health-related parameters and second attribute data comprising emotional-state parameters from a terminal device, and apply a statistical computation algorithm to the first attribute data to generate a body index, a risk index, and a lifestyle index;
generate a prompt sentence for a generative neural network model specifying instructions related to nutrient intake parameters, physical activity parameters, lifestyle adjustment parameters, and constraint parameters conditioned on the second attribute data, and transmit the prompt sentence to the generative neural network model to receive a natural language response;
parse the natural language response to extract a plurality of proposal datasets and convert the extracted proposal datasets into display data for transmission to the terminal device; and
generate a further prompt sentence based on the second attribute data and the analysis result, transmit the further prompt sentence to the generative neural network model, and dynamically update constraint control data based on a response from the generative neural network model.

19. The system according to claim 18, wherein the circuitry is configured to generate cooperation data from a first proposal dataset of the plurality of proposal datasets, transmit the cooperation data to an external goods delivery interface to enable selection of proposed item data, and receive order confirmation data from the external goods delivery interface.

20. A method performed by circuitry, the method comprising:
receiving, via a communication interface coupled to a packet-switched network, first attribute data comprising health-related parameters and second attribute data comprising emotional-state parameters from a terminal device, and storing the first attribute data and the second attribute data as structured data in a storage device;
applying a statistical computation algorithm to the stored first attribute data to generate an analysis result comprising a body index, a risk index, and a lifestyle index;
automatically generating, based on the analysis result, a prompt sentence for a generative neural network model, the prompt sentence specifying in natural language instructions related to at least nutrient intake parameters, substance ingestion parameters, physical activity parameters, lifestyle adjustment parameters, and constraint parameters conditioned on the second attribute data;
transmitting the prompt sentence to the generative neural network model via the communication interface, receiving a natural language response from the generative neural network model, and parsing the natural language response to extract at least a first proposal dataset, a second proposal dataset, a third proposal dataset, a fourth proposal dataset, and a constraint proposal dataset;
converting the extracted proposal datasets into display data and transmitting the display data to the terminal device; and
generating a further prompt sentence for the generative neural network model based on the second attribute data and the analysis result, transmitting the further prompt sentence to the generative neural network model, and dynamically updating constraint control data based on a response from the generative neural network model.
